(19) United States
(12) Patent Application Publication
(10) Pub. No.: US 2025/0000627 A1
(43) Pub. Date: Jan. 2, 2025

(54) APPARATUS FOR IRRIGATION, SUCTIONING AND OBTAINING SAMPLES OF THE ORAL CAVITY

(71) Applicant: SWIFTSURE INNOVATIONS INC., Pasadena (CA)

(72) Inventors: Deanne McCarthy, Pasadena (CA); Christopher David Gillespie, Toronto (CA); Kathryn Atwell, Halifax (CA); Joel Ironstone, Toronto (CA); Matthew Asselin, Toronto (CA); Rory Tatchell, Corner Brook (CA); Angie Hughes, Corner Brook (CA)

(73) Assignee: SWIFTSURE INNOVATIONS INC., Pasadena, NL (CA)

(21) Appl. No.: 18/700,192

(22) PCT Filed: Jul. 29, 2022
(86) PCT No.: PCT/CA2022/051162

§ 371 Date: Apr. 10, 2024

Related U.S. Application Data
(60) Provisional application No. 63/254,932, filed on Oct. 12, 2021.
(60) Provisional application No. 63/301,799, filed on Jan. 21, 2022.
(60) Provisional application No. 63/328,612, filed on Apr. 7, 2022.

(51) Int. Cl. A61C 17/02; A61B 10/00; A61M 1/00

(52) U.S. Cl. CPC A61C 17/0208; A61B 10/0051; A61C 17/0211; A61M 1/77; A61M 2210/0625

(57) ABSTRACT

Various embodiments are provided herein for a medical device that has: a suction element with an elongated arm for placement between a check and the upper and lower teeth of a patient, and at least one suction inlet configured to suction fluid out of the oral cavity; an irrigation element with a second plate for placement between the upper and lower teeth of the second side of the oral cavity and one or more irrigation outlets configured to irrigate the oral cavity with fluid; a suction port that is fluidically coupled to the suction element for providing suctioning of the oral cavity during use; and an irrigation port that is fluidically coupled to the irrigation element for providing irrigation to the oral cavity during use.

### CROSS-REFERENCE TO RELATED APPLICATION

This application claims the benefit of U.S. Provisional Patent Application No. 63/254,932 filed Oct. 12, 2021, U.S. Provisional Patent Application No. 63/301,799 filed Jan. 21, 2022 and U.S. Provisional Patent Application No. 63/328,612 filed Apr. 7, 2022; the entire contents of U.S. Provisional Patent Application No. 63/254,932, U.S. Provisional Patent Application No. 63/301,799 and United States Provisional Patent Application No. 63/328,612 are all hereby incorporated herein in their entirety.

### FIELD

Various embodiments are described herein of a medical device for suction and irrigation of an oral cavity in a patient.

### BACKGROUND

The following paragraphs are provided by way of background. They are not, however, an admission that anything discussed therein is prior art or part of the knowledge of persons skilled in the art.

A mechanically ventilated patient requires a tube to be inserted into their trachea so that a ventilator can deliver oxygen to their lungs. The tube may be an endotracheal tube, or a tube introduced via a tracheostomy. The proximal end of the tube that is outside of the patient's mouth is coupled to tubing that is attached to a ventilator so that air can be delivered to and received from the patient's lungs and the distal end of the tube is disposed below the patient's glottis where the trachea branches into the bronchial tubes. A cuff on the tube generally expands to engage with the patient's tracheal wall to thereby form a seal which permits ventilation while also preventing fluids and other material from being aspirated into the patient's lungs.

Mechanically ventilated patients require oral care periodically to prevent bacteria from proliferating in the oral cavity. Patients in palliative care, acute care, and other care environments are also completely dependent on their caregivers to provide oral care. This may be done by performing an irrigation process where fluid is distributed into the patient's mouth and removed by a suctioning process. Therefore, a device for appropriately irrigating and suctioning the patient's oral cavity with ease is needed.

### SUMMARY

Disclosed here are various configurations of a device for insertion into the mouth of a patient to allow for irrigation and/or suction to occur with ease as well as for one or more methods for use of the device.

In one broad aspect, in accordance with the teachings herein, there is provided at least one embodiment of a medical device for suction and irrigation of an oral cavity in a patient, the device comprising: a suction element configured to be placed at a first location of the oral cavity, the suction element comprising an elongated arm for placement between a cheek and the upper and lower teeth of the patient at the first location, the suction element further comprising at least one suction inlet configured to suction fluid out of the oral cavity; an irrigation element that is configured to be disposed at a second location of the oral cavity, the irrigation element comprising a second plate for placement between the upper and lower teeth of the second location of the oral cavity and one or more irrigation outlets configured to irrigate the oral cavity with fluid; a suction port that is fluidically coupled to the suction element for providing a negative pressure thereto for suctioning at least a first portion of the oral cavity during use; and an irrigation port that is fluidically coupled to the irrigation element for providing the fluid thereto for irrigating at least a second portion of the oral cavity during use.

In another broad aspect, in accordance with the teachings herein, there is provided at least one embodiment of a medical device for suction and irrigation of an oral cavity in a patient, the device comprising, the medical device comprising a suction element configured to be placed at a first location of the oral cavity, the suction element comprising a first plate for placement between upper and lower teeth at first location of the oral cavity and a paddle for placement between a cheek and the upper and lower teeth of the patient, the suction element further comprising at least one suction inlet configured to suction fluid out of the oral cavity; an irrigation element that is configured to be disposed at a second location of the oral cavity, the irrigation element comprising a second plate for placement between the upper and lower teeth of the second location of the oral cavity and one or more irrigation outlets configured to irrigate the oral cavity with fluid; a suction port that is fluidically coupled to the suction element for providing a negative pressure thereto for suctioning at least a first portion of the oral cavity during use; and an irrigation port that is fluidically coupled to the irrigation element for providing the fluid thereto for irrigating at least a second portion of the oral cavity during use.

In at least one embodiment, the device comprises a bridge that is adapted to couple the suction element to the irrigation element.

In at least one embodiment, the bridge comprises a top edge and a bottom edge, the top edge being tapered towards the bottom edge.

In at least one embodiment, the bottom edge of the bridge is tapered towards the top edge.

In at least one embodiment, a center portion of the bridge has a low profile relative to outer portions of the bridge.

In at least one embodiment, the bridge comprises a top surface and a bottom surface that are substantially flat and spaced apart from one another by the same distance in their lateral extent from the suction element to the irrigation element.

In at least one embodiment, the suction port and the irrigation port are located at the bridge and may be laterally offset from a center of the bridge and are configured to extend outside of the oral cavity during use.

In at least one embodiment, the paddle of the suction element comprises an elongated arm with an enlarged head.

In at least one embodiment, the paddle of the suction element comprises an elongated arm.

In at least one embodiment, the suction element comprises a paddle formed on the elongated arm and disposed adjacent a distal end of the suction element.

In at least one embodiment, the paddle has a raised surface on an inner surface thereof.

In at least one embodiment, at least one suction inlet near and spaced apart from the distal end of the raised surface of the suction element.

In at least one embodiment, the paddle comprises a disk located at an interior surface of the paddle, the disk containing the at least one suction inlet.

In at least one embodiment, the device further comprises a clip that is adapted to be releasably coupled to the suction port and the irrigation port and located exterior to the oral cavity during use, wherein the clip is configured to control a distance between inner edges of the suction element and the irrigation element when the device is placed within the oral cavity.

In at least one embodiment, the clip comprises a first slot that is configured to slidably receive the suction port, a second slot that is configured to slidably receive an endotracheal tube, dental instrument or other medical instrument, and a third slot that is configured to slidably receive the irrigation port.

In at least one embodiment, the suction port and the irrigation port each comprise a positioning slot for releasably receiving the clip where the positioning slot is located at a distance away from a front surface of the bridge and the first and third slots of the clip are located to allow the suction port and the irrigation port to be moved together when the clip is placed at the positioning slots thereby widening a distance between inner edges of the suction element and the irrigation element.

In at least one embodiment, a suction line is fluidly coupled to the suction port.

In at least one embodiment, an irrigation line is fluidly coupled to the irrigation port.

In at least one embodiment, the irrigation line comprises a distal connector to fluidly couple to an external irrigation device.

In at least one embodiment, the suction line comprises a distal connector to fluidly couple to an external suction device.

In at least one embodiment, the second plate of the irrigation element comprises a top surface, a bottom surface, and an edge around an outer border of the top surface and the bottom surface, and the irrigation outlets are located proximally to the edge on at least one of the top surface and the bottom surface of the second plate of the irrigation element.

In at least one embodiment, the second plate of the irrigation element comprises a top surface, a bottom surface, and an edge around an outer border of the top surface and the bottom surface, and the irrigation outlets are located on at least one of the top surface and the bottom surface of the irrigation element.

In at least one embodiment, the device comprises an interior irrigation channel that is located within the irrigation element and has a first end that is fluidly coupled to the irrigation port and a second end that is fluidically coupled to the one or more irrigation outlets.

In at least one embodiment, the device comprises an interior suction channel within the suction element has a first end that is fluidly coupled to the suction port and a second end that is fluidically coupled to the at least one suction inlet.

In at least one embodiment, the medical device further comprises a locking mechanism that is adapted to be releasably coupled to the suction port and the irrigation port and exterior to the oral cavity during use, wherein the locking mechanism comprises a first arm having at least one clip and a second arm having at least one ridge, the at least one ridge being releasably engaged by the at least one clip and configured to control a distance between the suction element and the irrigation element when the device is placed within the oral cavity.

In at least one embodiment, the locking mechanism comprises first, second and third ridges on the second arm for releasable engagement by the at least one clip on the first arm, the first ridge being located to allow the suction port and the irrigation port to be moved apart when the at least one clip is placed at the first ridge thereby narrowing a distance between the suction element and the irrigation element and the third ridge being located to allow the suction port and the irrigation port to be moved together when the at least one clip is placed at the third ridge thereby widening the distance between the suction element and the irrigation element.

In at least one embodiment, the medical device further comprises a handle mechanism that is adapted to be releasably coupled to the suction port and the irrigation port and located exterior to the oral cavity during use, wherein the handle mechanism comprises a male connector and a female connector.

In at least one embodiment, the male and female connectors include first and second tabs, respectively, and the first tab and the second tab are releasably engaged when pressed together to control a distance between the suction element and the irrigation element when the device is placed within the oral cavity.

In at least one embodiment, the female connector includes a slot sized and shaped to accommodate a post of the male connector and during use the male and female connectors are releasably engaged when the male connector is slid into the female connector to connect the suction element and the irrigation element.

In at least one embodiment, the male and female connectors are first and second tabs, respectively, that are configured to connect the suction element and the irrigation element by a button snap, buckle, Velcro, double sided adhesive, screws or a pin.

In at least one embodiment of a medical device described herein, the at least one suction element comprises a rectangular shaped slot, a conical shaped slot, an elliptical shaped slot, a semi-circular shaped slot, an almond-shaped slot, a curved slot or a curved slit.

In another aspect, in accordance with the teachings herein there is provided at least one embodiment of a kit for irrigation of an oral cavity of a patient, the kit comprising: a medical device defined in accordance with any one of the embodiments described herein; and a medical instrument for placement within the oral cavity of the patient.

In at least one embodiment, the medical instrument of the kit is an endotracheal tube, a dental instrument or a disposable oral care product.

In another aspect, in accordance with the teachings herein there is provided at least one embodiment of a method for irrigating and suctioning an oral cavity in a patient, the method comprising: placing a medical device within the oral cavity of the patient, the medical device being defined in accordance with any one of embodiments described herein; irrigating the oral cavity with fluid delivered using the irrigation element of the medical device; and suctioning the fluid or any other liquids out of the oral cavity using the suction element of the medical device.

In at least one embodiment, the method further comprises receiving, at the bridge of the medical device, an endotracheal tube.

In at least one embodiment, the method further comprises releasably coupling the suction port and the irrigation port of the medical device to a clip configured to control a distance between the suction element and the irrigation element when placing the medical device within the oral cavity.

In at least one embodiment, the method further comprises fluidly coupling the suction port to a suction line.

In at least one embodiment, the method further comprises fluidly coupling, by a distal connector, the suction line to an external suction device.

In at least one embodiment, the method further comprises fluidly coupling the irrigation port to an irrigation line.

In at least one embodiment, the method further comprises fluidly coupling, by a distal connector, the irrigation line to an external irrigation device or a fluid-filled bag that is adapted to provide fluid through gravity or is coupled to a pressurized system to provide the fluid for irrigation during use.

In at least one embodiment, prior to providing irrigation, the method further comprises positioning the patient such that a head of the patient is in a lower position relative to feet of the patient.

In at least one embodiment, the method further comprises positioning a torso of the patient on a side so that the suction element is disposed in a buccal pocket located in a lowest position of the oral cavity.

In at least one embodiment, the method further comprises instilling fluid into a nostril of the patient.

In at least one embodiment, the method further comprises obtaining at least one oral sample using the medical device when providing suctioning to the at least second portion of the oral cavity.

In at least one embodiment, the method comprises obtaining the at least one oral sample without providing irrigation, after providing irrigation or during providing irrigation.

In at least one embodiment, the method comprises using a syringe to provide the suctioning so that the at least one oral sample is contained in a portion of the syringe.

In at least one embodiment, the method comprises using a sample container that is coupled to the suction line of the device for receiving the at least one oral sample when suctioning is provided to the at least second portion of the oral cavity of the patient.

In another aspect, in accordance with the teachings herein there is provided a use of a medical device for irrigating, suctioning and/or obtaining at least one sample from an oral cavity in a patient wherein the medical device is defined according to any one of the embodiments described herein, wherein the medical device is adapted to perform any one of the methods described herein during the use.

In another aspect, in accordance with the teachings herein there is provided at least one embodiment of a medical device for suction and irrigation of an oral cavity in a patient, the device comprising: a suction element configured to be placed at one side of the oral cavity, the suction element comprising an elongated arm for placement between a cheek and the upper and lower teeth of the patient, the suction element further comprising at least one suction inlet configured to suction fluid out of the oral cavity; an irrigation element that is configured to be disposed at a second side of the oral cavity, the irrigation element comprising a plate for placement between the upper and lower teeth of the second side of the oral cavity and one or more irrigation outlets configured to irrigate the oral cavity with fluid; a suction port that is fluidically coupled to the suction element for providing a negative pressure thereto for suctioning the oral cavity during use; an irrigation port that is fluidically coupled to the irrigation element for providing the fluid thereto for irrigating the oral cavity during use, and a locking mechanism that is adapted to be releasably coupled to the suction port and the irrigation port and exterior to the oral cavity during use, wherein the locking mechanism comprises a first arm having at least one clip and a second arm having at least one ridge, the at least one ridge being releasably engaged by the at least one clip and configured to control a distance between the suction element and the irrigation element when the device is placed within the oral cavity.

In another aspect, in accordance with the teachings herein there is provided at least one embodiment of a medical device for suction and irrigation of an oral cavity in a patient, the device comprising: a suction element configured to be placed at one side of the oral cavity, the suction element comprising an elongated arm for placement between a cheek and the upper and lower teeth of the patient, the suction element further comprising at least one suction inlet configured to suction fluid out of the oral cavity; an irrigation element that is configured to be disposed at a second side of the oral cavity, the irrigation element comprising a plate for placement between the upper and lower teeth of the second side of the oral cavity and one or more irrigation outlets configured to irrigate the oral cavity with fluid; a suction port that is fluidically coupled to the suction element for providing a negative pressure thereto for suctioning the oral cavity during use; an irrigation port that is fluidically coupled to the irrigation element for providing the fluid thereto for irrigating the oral cavity during use, and a handle mechanism that is adapted to be releasably coupled to the suction port and the irrigation port and exterior to the oral cavity during use, wherein the handle mechanism comprises a male connector and a female connector.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a perspective view of an example embodiment of a medical device for suction and irrigation of an oral cavity.

FIGS. 2 and 3 are top and bottom views, respectively, of the medical device of FIG. 1.

FIGS. 4A and 4B are right and left side views, respectively, of the medical device of FIG. 1, where FIG. 4A shows the irrigation and suction elements and FIG. 4B shows an outer side of the suction element.

FIG. 5 is a front perspective view showing another example embodiment of a medical device with a releasably couplable clip.

FIG. 6 is a partial top perspective view of the suction element of the medical devices of FIGS. 1-5.

FIG. 7A is a partial top view of an example embodiment of an irrigation element that may be used with the medical devices of FIGS. 1 to 6.

FIG. 7B is a partial right-side view of the irrigation element of FIG. 7A, showing the direction of fluid flow out of irrigation outlets.

FIGS. 8A-8C show a front perspective view, a front view and a top view, respectively, of an example embodiment of a clip that may be used with the medical devices of FIGS. 1-7B.

FIGS. 9A-9C show front views of various example embodiments of the clip of FIGS. 8A-8C.

FIGS. 10 and 11 show front and side views, respectively, of a patient using a medical device of the teachings herein without the use of a clip.

FIGS. 12 and 13 show front and side views, respectively, of a patient using the medical device of the teachings herein with the use of the clip.

FIGS. 14 and 15 show a top perspective view and a partial top view of another example embodiment of a medical device with a clip that comprises a locking mechanism to control the movement of the medical device.

FIG. 16 shows a partial top perspective view of another example embodiment of a medical device with another locking mechanism to control the movement of the medical device.

FIGS. 17A and 17B show partial top perspective views of the medical device of FIG. 16 with the locking mechanism moved to different positions.

FIG. 18 shows a partial top perspective view of another example embodiment with another locking mechanism to control the movement of the medical device.

FIGS. 19A and 19B show partial top views of the medical device of FIG. 18 with the locking mechanism moved to different positions.

FIG. 20 shows a front perspective view of another example embodiment of a medical device for suction and irrigation of an oral cavity.

FIGS. 21A and 21B show a partial top view and a cross-sectional partial right-side view, respectively, of the suction element of the medical device of FIG. 20.

FIG. 21C shows a top view of a horizontal cross section of the medical device of FIG. 20 without the irrigation or suction lines.

FIG. 22 is a perspective view of another example embodiment of a medical device for suction and irrigation of an oral cavity.

FIGS. 23A and 23B are front and back views, respectively, of the medical device of FIG. 22, where FIG. 23A shows the suction and irrigation ports and FIG. 23B shows the suction and irrigation elements.

FIGS. 24A and 24B are right and left side views, respectively, of the medical device of FIG. 22, where FIG. 24A shows the irrigation and suction elements and FIG. 24B shows an outer side of the suction element.

FIG. 25 shows a top view of the medical device of FIG. 22.

FIG. 26 is a perspective view of another example embodiment of a medical device for suction and irrigation of an oral cavity.

FIGS. 27A and 27B are front and back perspective views, respectively, of the medical device of FIG. 26, where FIG. 27A shows the suction and irrigation lines and FIG. 27B shows the suction and irrigation elements.

FIGS. 28A and 28B are right and left side views, respectively, of the medical device of FIG. 26, where FIG. 28A shows the irrigation and suction elements and FIG. 28B shows an outer side of the suction element.

FIGS. 29A and 29B show a partial top view and a cross-sectional partial right side view, respectively, of the suction element of the medical device of FIG. 26.

FIG. 30 is a perspective view of another example embodiment of a medical device for suction and irrigation of an oral cavity.

FIG. 31 is perspective view of another example embodiment of a medical device for suction and irrigation of an oral cavity with a locking mechanism to control the movement of the medical device.

FIGS. 32A and 32B show partial perspective views of the medical device of FIG. 31 with the locking mechanism in disengaged and engaged configurations, respectively.

Further aspects and features of the example embodiments described herein will appear from the following description taken together with the accompanying drawings.

### DETAILED DESCRIPTION OF THE EMBODIMENTS

The headings and Abstract of the Disclosure provided herein are for convenience only and do not interpret the scope or meaning of the embodiments.

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors, or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical, structural or fluidic connotation. For example, as used herein, the terms coupled or coupling can indicate that two elements or devices can be directly connected to one another or connected to one another through one or more intermediate elements or devices via a mechanical element, a structural element, a gas flow or a fluid flow depending on the particular context.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, 10%, 15% or 20%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, 10%, 15% or 20%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

As mentioned mechanically ventilated patients require a tube to be inserted into the trachea so that a ventilator can deliver oxygen to the lungs. The tube is generally an endotracheal tube, or a tube introduced via a tracheostomy. The proximal end of the tube is coupled to tubing attached to a ventilator so that air can be delivered to and from the lungs and the distal end of the tube is disposed below the glottis into the trachea.

In accordance with the teachings herein, there are provided various example embodiments of medical devices that may be used to routinely cleanse the areas of the oral cavity that may otherwise serve as a reservoir for pathogens which may prevent or dramatically slow the formation of biofilms that contain pathogenic microorganisms.

Accordingly, the various devices and associated methods described herein may be used for providing improved oral care compared to conventional devices and techniques. The various devices described herein may be used to reduce the length for which mechanical ventilation is needed since the various devices described herein may be used to remove bacteria and organic debris from the oral cavity, the two main reservoir for various pathogens that can damage/impair a patient's respiratory system. These various devices may allow for irrigation and suctioning of irrigation fluid without the required use of external suction catheters, which decreases risk of mucous membrane injury and may promote healthy mucous membranes. The devices may also allow for irrigation without dwell time, meaning that solution can be introduced into the patient's oral cavity and simultaneously suctioned out. Such devices and methods may be cost effective, efficient, and easy to use, allowing for frequent intervention.

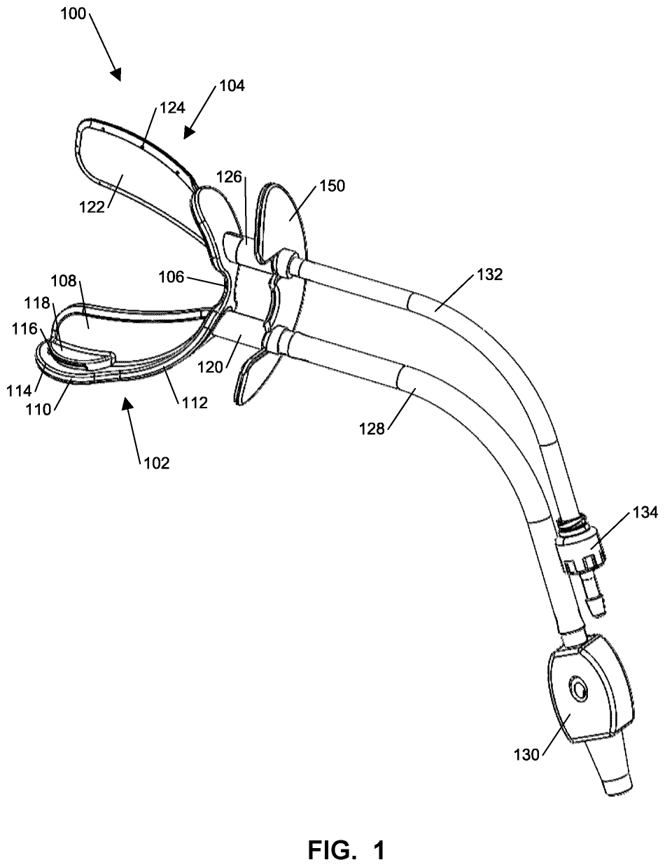

Referring now to FIG. 1, a top perspective view of an example embodiment of a medical device 100 in accordance with the teachings herein is shown. The medical device 100 may be used in conjunction with an endotracheal tube (not shown) for irrigation and suctioning of an oral cavity of a patient. However, the medical device 100, which may also be referred to as an oral appliance, may be used without an endotracheal tube. The medical device 100 includes two portions, where a first portion includes a suction element 102 and a second side portion includes an irrigation element 104. The suction element 102 and the irrigation element 104 are coupled to one another by a bridge 106. Each of elements 102 and 104 is designed to be placed within certain locations of an oral cavity of a patient. For example, the suction element 102 may be configured to be placed at a first location (e.g., at the right side) of the oral cavity of a patient while the irrigation element 104 is placed at a second location (e.g., at the left side) of the oral cavity of the patient. In an alternative embodiment, the suction element 102 may be configured to be placed at the left side of the oral cavity of a patient while the irrigation element 104 is placed at the right side of the oral cavity of the patient. In an alternative embodiment, the medical device 100 may be configured so as to allow for the medical device 100 to be flipped easily and the suction element 102 to be placed on either the left or right side of the oral cavity.

In at least one embodiment, as shown in FIG. 1, the suction element 102 may comprise a first plate 108 and a paddle (or side wall) 110. The first plate 108 may be flat or may have a slight contour or curvature. The paddle 110 may comprise an elongated arm 112 and an enlarged head 114. However, in at least one alternative embodiment, the enlarged head 114 is not used but rather the suction element is an elongated arm with a more uniform shape. The first plate 108 of the suction element 102 may be used for placement between the upper and lower teeth at one side of the patient's mouth. The first plate 108 preferably has smooth edges and may be rectangular, square, rectangular with rounded edges, oval, circular, or any other shape that may be placed within the patient's mouth to fit between the upper and lower teeth of the patient. The paddle 110 may be placed on the outer side of the upper and lower teeth of the patient such that the elongated arm 112 and the enlarged head 114 of the paddle 110 are located between the outer surface of the patient's upper and lower teeth and the inner surface of the cheek of the patient's oral cavity. The paddle 110 preferably has smooth edges and may be rectangular, square, rectangular with rounded edges, oval, circular, or any other shape that may be placed within the patient's mouth to fit between the teeth and the cheek of the patient.

The suction element 102 comprises at least one suction inlet 116. The suction inlet 116 is for suctioning fluid out from at least a first portion of the oral cavity. As further described in FIG. 6, the enlarged head 114 of the paddle 110 may have a disk 118 or raised surface on its inner surface. The disk 118 extends inwardly and away from the inner surface of the enlarged head 114. The disk 118 may be raised from the surface of the paddle 110 so as to ensure a space between the suction inlet 116 and a baffle (i.e., a point on the suction element 102) where the paddle 110 comes in contact with the cheek tissue of the user. The suction inlet 116 of the suction element 102 may be defined by an opening formed on a top portion of the disk 118. In alternative embodiments, there may be more than one suction inlet 116 that is included in the suction element 102 or the suction inlet may be formed on another portion of the disk 118. In various embodiments, suction apertures may have any shape, size, location, or number along the suction element.

The suction element 102 further contains a suction port 120 protruding from an outer surface of the suction element 102 that faces away from the patient's oral cavity in use. In at least one embodiment, the suction port 120 is located approximately on a front portion of the suction element 102 that may be located on the outside of the patient's mouth when the medical device 100 is in use. In at least one embodiment, the suction port 120 may be located on the bridge 106 or laterally offset from the bridge 106. The suction port 120 may protrude from the suction element 102 by any suitable distance such that it may be connected to a physical element that provides suction such as a suction line or syringe.

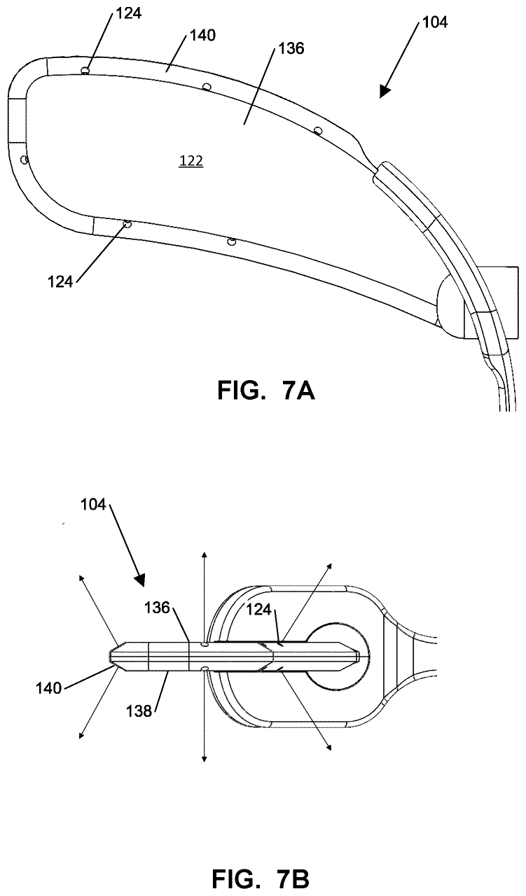

The irrigation element 104 of the medical device 100 comprises a second plate 122. The second plate 122 is for placement on the opposite side of the oral cavity and may be positioned between the teeth and/or against the cheek of patient, on the side opposite to that of the first flat plate 108 of the suction element 102. The second plate 122 may be flat or may have a slight contour or curvature. The second plate 122 may preferably has smooth edges and be rectangular, square, rectangular with rounded edges, oval, circular, or any other shape that may be slid between or placed within the teeth of a patient. The second plate 122 of the irrigation element 104 comprises one or more irrigation outlets 124. The irrigation outlets 124 allow a fluid to be discharged or irrigated directly into at least a second portion of the oral cavity. The irrigation outlets 124 are further discussed in relation to FIGS. 7A-7B. The irrigation element 104 further contains an irrigation port 126 protruding from an outer surface of the irrigation element 104 that faces away from the patient's oral cavity in use and may be exterior to the patient's mouth during use. In at least one embodiment, the irrigation port 126 is located approximately on the front of the irrigation element 104. In at least one embodiment, the irrigation port 126 may be located on the bridge 106 or laterally offset from the bridge 106. The irrigation port 126 may protrude from the irrigation element 104 by any suitable distance such that it may be connected to a physical element that provides suction such as a suction line or syringe.

As mentioned, the suction element 102 and the irrigation element 104 of the medical device 100 may be coupled to one another by a bridge 106. As can be seen in FIG. 1, the bridge 106 connecting the suction element 102 and the irrigation element 104 may be tapered in the middle. The top edge of the bridge 106 may be tapered downwards relative to the upper edges of the medical device 100 to allow for, in at least one embodiment, to receive an endotracheal tube to be located at an upper surface there unimpeded by the medical device 100. In at least one embodiment, the bottom edge of the bridge 106 may be tapered upwards away from bottom edges of the medical device 100 to be easily placed at the inner surface of the bottom lip of the patient. The medical device 100 may be, in at least one embodiment, configured as to be easily flipped over within the oral cavity of the patient to allow for suctioning on either side of the mouth. As such, in at least one embodiment, the bridge 106 may be tapered on the top edge and the bottom edge so the endotracheal tube may be placed appropriately no matter the orientation of the medical device 100.

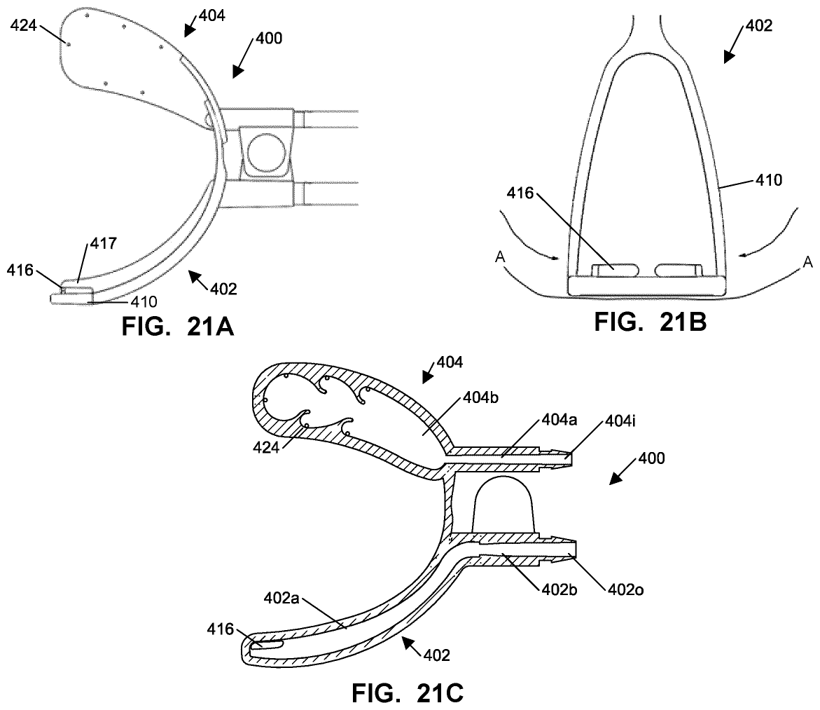

In at least one embodiment, an end of the suction port 120 of the medical device 100 is fluidly coupled to a suction line 128. A second end of the suction portion 120 is fluidically coupled to an interior suction channel that is formed within the suction element 102 (an example of this for another embodiment is shown in FIG. 21C). The suction line 128 may further comprise a first distal connector 130 that may be fluidly coupled to an external suction device, such as a wall mounted suction device, or any other suction device. The suctioning may be activated by a switch or active thumb actuation on the first distal connector 130 of the suction line 128. In at least one embodiment, a first end of the irrigation port 126 is fluidly coupled to an irrigation line 132. A second end of the irrigation port 126 is fluidically coupled to an interior irrigation channel that is formed within the irrigation element 104 (an example of this for another embodiment is shown in FIG. 21C). The irrigation line 132 may further comprise a second distal connector 134 that may be fluidly coupled to an external irrigation device, such as a syringe, a bag of saline within a pressure bag, or any other device used to irrigate the oral cavity of a patient. In at least one embodiment, the irrigation line may be coupled to a fluid-filled bag that is adapted to provide fluid through gravity or is coupled to a pressurized system.

In another embodiment, the suction port 120 and the irrigation port 126 may be a single port. The single port may be located along any surface of the medical device 100. In such embodiment, the fluid used to irrigate the oral cavity may be introduced to the oral cavity through the same port as it is suctioned out. In such embodiments, the single port may be fluidly coupled to a fluid line used for both irrigation and suction. The fluid line may be distally connected to a device that is able to alternate between providing fluid for irrigation and then removing the fluid from the patient's oral cavity by applying suction.

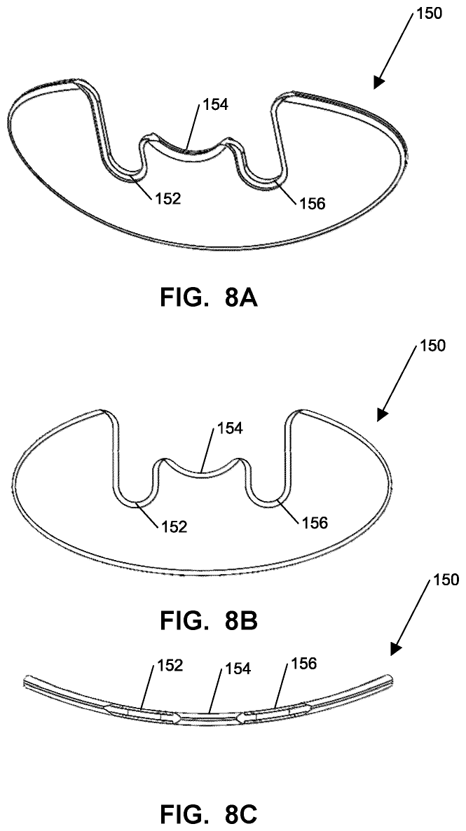

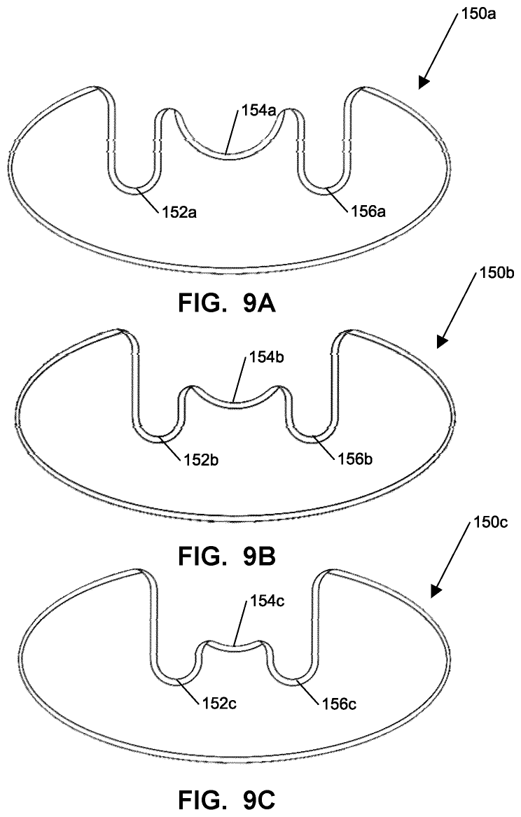

In at least one embodiment, the medical device 100 may further include a clip 150 (see FIGS. 8A-9C). The clip 150 may be releasably coupled to the suction port 120 and the irrigation port 126. The clip 150 may be configured to sit at the exterior of the oral cavity of the patient. The clip 150 may be used to urge the suction element 102 and irrigation element 104 closer together when they are placed in a patient's mouth and then optionally farther apart in order to accommodate (i.e., fit) a larger sized mouth in which the two quadrants of a patient's lower (upper) teeth are spaced further apart from one another.

The medical device 100 may be formed of any number of materials such as soft resilient materials like silicone, polyurethane, or other materials known in the art. For example, the medical device 100 may be formed of a flexible material to allow for the suction element 102 and the irrigation element 104 of the medical device 100 to be movable with respect to the bridge 106. The flexibility of the material may allow the medical device 100 to be easier to insert into the mouth of the patient, as the suction element 102 and the irrigation element 104 may be moved such that they are closer together to allow for easier insertion into the oral cavity as the medical device 100 may be laterally narrower. Once the medical device 100 is inserted into the oral cavity of the patient, the medical device 100 may “expand” back to the original configuration before use where the suction element 102 and the irrigation element 104 move away from one another. In at least one embodiment, the suction element 102 and the irrigation element 104 may be formed of a non-flexible (i.e., rigid) material and the bridge 106 may be formed of a flexible material. The flexibility of the bridge 106 may still allow the medical device 100 to be inserted into the mouth of the patient easily while the rigidity of the suction element 102 and irrigation element 104 may help retain the structure of the medical device 100. Alternatively, in at least one embodiment, the medical device 100 may be formed of any combination of a non-flexible (i.e., rigid) material and/or a flexible material.

In at least one embodiment, the medical device 100 may have a depth of approximately 40 mm, where the depth is characterized by the distance from the bridge 106 of the medical device 100 to the ends of the suction element 102 and the irrigation element 104 (i.e., the ends of the suction element 102 and the irrigation element 104 that reach the furthest into the patient's oral cavity). Alternatively, in at least one embodiment, the medical device 100 may have a depth smaller than about 40 mm for use with patients that have smaller oral cavities. Alternatively, in at least one embodiment, the medical device 100 may have a depth larger than about 40 mm for use with patients that have larger oral cavities.

In at least one embodiment, the medical device 100 may have a width of approximately 85 mm, where the width is characterized by the distance from the furthest outer edge of the suction element 102 relative to the bridge 106 to the furthest outer edge of the irrigation element 104 relative to the bridge 106. In at least one embodiment, the medical device 100 may have a width of less than about 85 mm for use with patients that have smaller oral cavities. In at least one embodiment, the medical device 100 may have a width greater than about 85 mm for use with patients that have larger oral cavities. In any of the disclosed embodiments, the medical device 100 may be compressed laterally by a certain amount, such as approximately 15 mm, for example.

In at least one embodiment, the first and second plates 108 and 122 may have a narrower width, where the width is characterized by the distance from an outer edge of a given plate to an inner edge of the given plate. The narrow width may be useful for a smaller embodiment directed towards the use of children or those with a smaller anatomy in the jaw. In at least one embodiment, the first and second plates 108 and 122 may have a wider width. The wider width may be of a larger embodiment that is directed towards adult use or use for those with a larger anatomy in the jaw. In at least one embodiment, the suction inlet is disposed at least a distance of about 3 mm from the baffle (i.e., a point on the suction element) where there is contact with the cheek tissue of the patient.

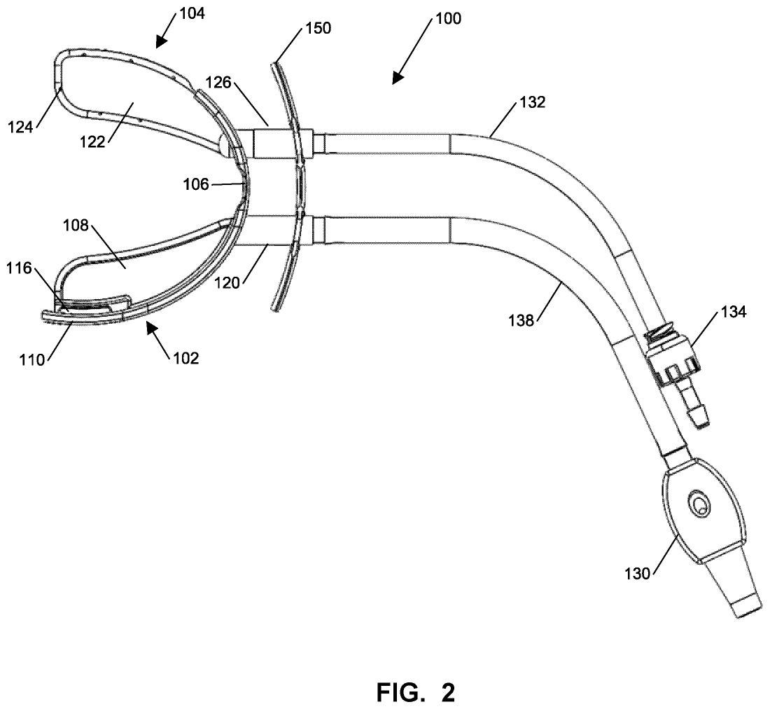

FIGS. 2 to 5 show the suction port 120 being fluidly connected to the suction line 128 and the irrigation port 126 being fluidly connected to the irrigation line 132. FIGS. 2 and 3 further show an example embodiment of the suction element 102, where the suction inlet 116 may be located on a disk 118 along the inner surface of the enlarged head 114 of the paddle 110. In this example embodiment, the suction element 102 may have two suction inlets 116, where a first suction inlet is located along the top of the disk 118 and a second located suction inlet is located along the bottom of the disk 118. FIGS. 2 and 3 also show an example embodiment of the irrigation element 104 in which irrigation outlets 124 may be located near an outer edge, a rear edge and an inner edge or an inner region away from any edges of the second plate 122 of the irrigation element 104.

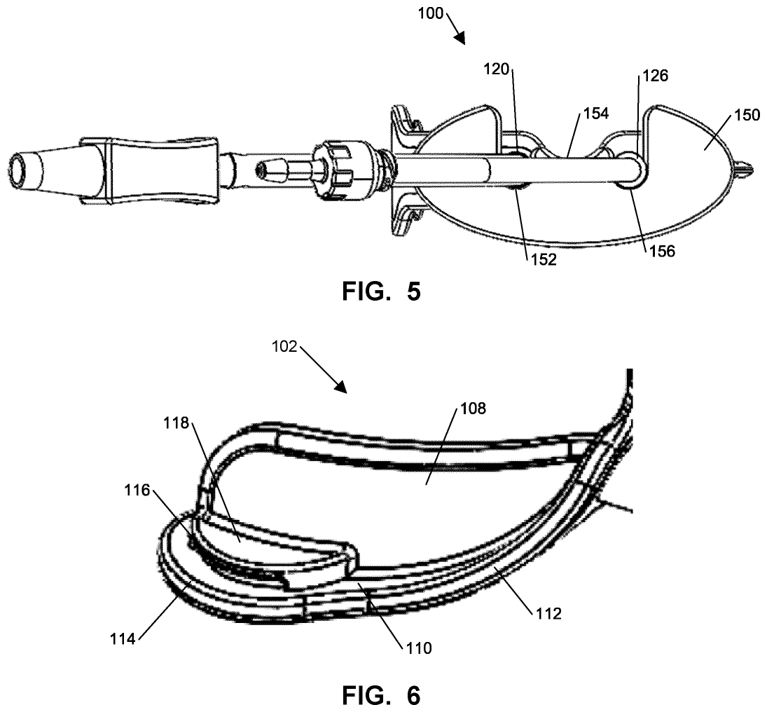

FIG. 5 shows a perspective view of the clip 150 in one example embodiment. The clip 150 may comprise a first slot 152 for releasably receiving a portion of the suction port 120, a second slot 154 for releasably receiving a portion of an endotracheal tube (not shown), and a third slot 156 for releasably receiving a portion of the irrigation port 126. As shown in FIG. 5, the second slot 154 of the clip 150 and the taper of the bridge 106 of the medical device 100 may align in a frontal upwards direction. The second slot 154 of the clip 150 and the top edge taper of the bridge 106 may align so as to create an unimpeded path for the endotracheal tube when the device 100 is interested into the mouth of a patient that is currently intubated. The endotracheal tube may be in use before insertion of the medical device 100 or may be inserted into the patient's oral cavity after the medical device 100 has been inserted into the patient's oral cavity.

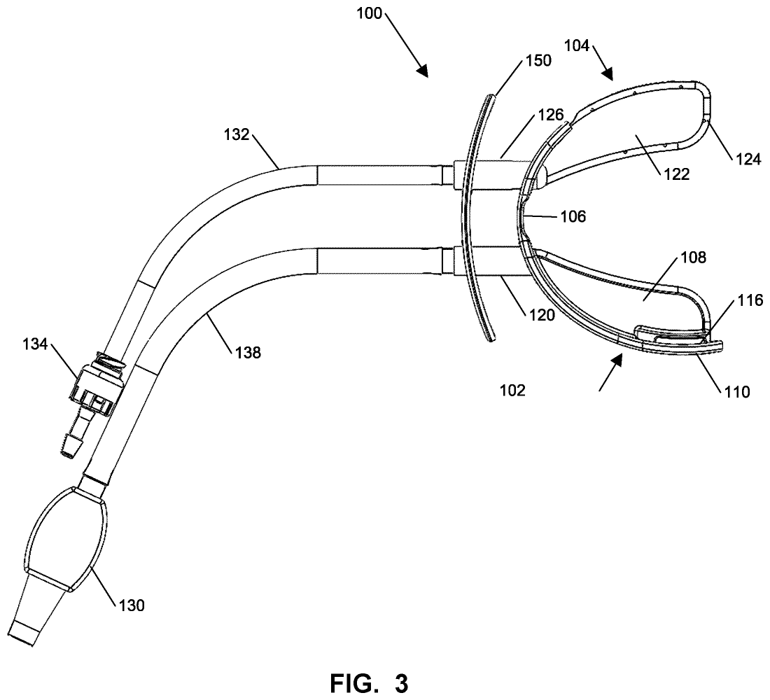

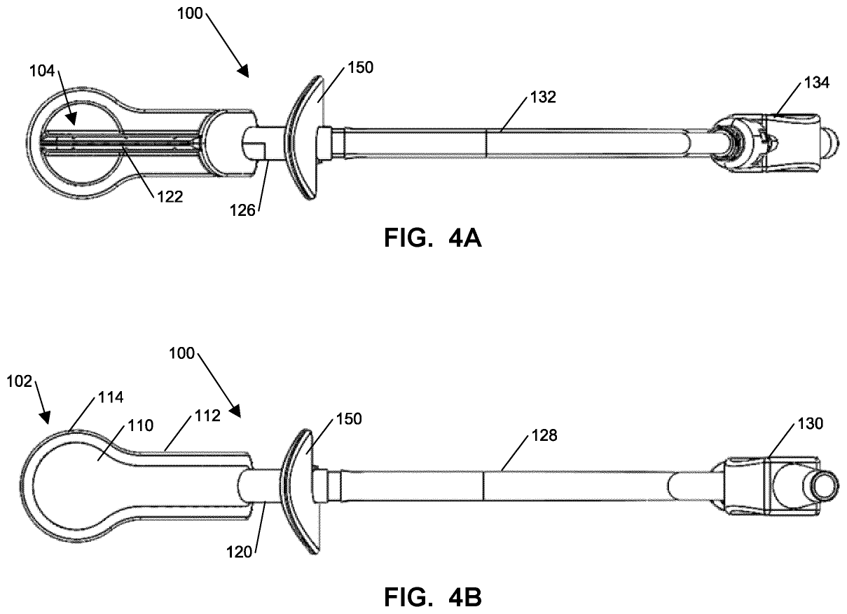

FIG. 6 shows a partial top perspective view of the suction element 102 of the medical device 100 of FIGS. 1 to 5. Shown in FIG. 6 is the disk 118 along the inner surface of the enlarged head 114 of the paddle 110 of the suction element 102. At least one suction inlet 116 is located on the disk 118. In at least one embodiment, multiple suction inlets 116 are disposed along the disk 118. In at least one embodiment, the suction inlet 116 is located on the top of the disk 118 (as shown in FIG. 6) as well as on the bottom of the disk 118 (as shown in FIG. 4B). Alternatively, in at least one embodiment, the paddle 110 does not include a disk 118, and the at least one suction inlet 116 is located on and/or under the first plate 108 of the suction element 102. In such embodiments, there may be multiple suction inlets of any size located around the perimeter and/or central region of the first plate 108. In at least one embodiment, the at least one suction element 116 may be located on the paddle 110 of the suction element 102. In said embodiments, there may be multiple suction inlets 116 of any size located on the elongated arm 112 or the enlarged head 114 of the paddle 110.

The suction inlet 116 of the suction element 102 is fluidly connected to the suction port 120 by a suction channel. This suction channel may allow for the fluid suctioned from the oral cavity of the patient to be removed more easily and disposed of through the suction port 120 and the suction line 128 to a wall mounted suction device, for example.

In at least one embodiment, the patient may be positioned on their side to create a low point in the patient's oral cavity where the irrigation fluid can accumulate. The medical device 100 may be placed within the oral cavity such that the suction element 102 is within the buccal pocket between the teeth and cheek of the patient. The low point of the medical device 100 in the oral cavity may also be in the buccal pocket. This may allow for easier suctioning of the irrigation fluid out of the oral cavity. In some cases, the disk 118 of the suction element 102 may be used to prevent mucous membranes from invaginating into the suction inlet 116. For example, the enlarged head 114 of the paddle 110 of the suction element 102 may contact the mucous membrane of the oral cavity of the patient. As the suction inlet 116 is located on the disk 118 on the inner surface of the enlarged head 114, there is a protective component separating the mucous membrane of the patient from the suction inlet 116. The disk 118 allows suction to occur in the oral cavity without the mucous membrane impeding suction or damaging the patient.

Referring now to FIGS. 7A and 7B, shown therein are partial views of example embodiments of the irrigation outlets 124 of the medical device 100 of FIGS. 1 to 5. FIG. 7A shows a partial top view of an irrigation element 104 with a second plate 122. FIG. 7B shows a partial right-side view of the irrigation element 104. The second plate 122 comprises a top surface 136, a bottom surface 138, and a tapered edge 140 around the outer border of the top surface 136. In at least one embodiment, the irrigation outlets 124 may be placed on the top surface 136 around the tapered edge 140 of the second plate 122, as shown in FIG. 7A. Alternatively, in at least one embodiment, as shown in FIG. 7B, the irrigation outlets 124 may be located along the tapered edge 140 of the second plate 122 on both the top surface 136 and the bottom surface 138. In another alternative, in at least one embodiment, the irrigation outlets 124 may be interspersed throughout the top surface 136. In yet another alternative, in at least one embodiment, the irrigation outlets 124 may be interspersed throughout the bottom surface 138. In yet another alternative, in at least one embodiment, the irrigation outlets 124 may be interspersed throughout both the top surface 136 and the bottom surface 138. In any of the previously disclosed embodiments, the irrigation outlets 124 may be placed so as to allow for irrigation fluid to be ejected/sprayed along: (1) an outer surface of the teeth, (2) between the teeth and the inner surface of the cheek of the patient, (3) between lower and/or upper surfaces of the upper teeth and lower teeth side, and/or (4) between the inner surface of the teeth and the tongue of the patient. Further shown in FIG. 7B is the direction of fluid flow from the irrigation outlets 124 (as shown by the arrows).

The irrigation outlets 124 of the irrigation element 104 are fluidly connected to the irrigation port 126 by a fluid channel (an example of this is shown for another embodiment in FIG. 21C). The fluid channel allows for fluid from a syringe, for example, or any other fluid dispersing device/fluid source, to be introduced into the oral cavity of the patient. In at least one embodiment, there may be multiple smaller fluid channels travelling from the inner edge of the irrigation port 126 to each of the irrigation outlets 124 individually. In another embodiment, the channel may include a single fluid channel travelling, for example, around the tapered edge 140 of the irrigation element 104 to each or the irrigation outlets 124, wherein the fluid channel may have a fenestration at each of the irrigation outlets 124 to allow for fluid to be discharged into the patient's oral cavity and this channel then follows a return path back to the irrigation port 126.

The suction inlet 116 and irrigation outlets 124 may allow for simultaneous introduction of a fluid into the patient's mouth and removal of the fluid from the patient's mouth, thereby reducing dwell time of the fluid within the mouth of the patient. The simultaneous introduction of fluid while suctioning out fluid may allow for a high amount of fluid to be irrigated and subsequently suctioned through the mouth of the patient when the irrigation flow and suction flow are about equal. Accordingly, a majority or about all of the fluid may be able to be rinsed through the mouth without having the fluid be translocated from the oral cavity down the throat of the patient.

Referring now to FIGS. 8A to 8C, FIG. 8A shows a perspective view of a clip 150, FIG. 8B shows a front view of the clip 150 and FIG. 8C shows a top view of the clip 150. As disclosed above, the clip 150 may include a first slot 152 for receiving a suction port 120 of a medical device described herein, a second slot 154 for receiving an endotracheal tube, dental instrument, and/or other medical instrument, and a third slot 156 for receiving an irrigation port 126 of a medical device described herein.

In at least one embodiment, the clip 150 is releasably coupled to the suction port 120 and the irrigation port 126 of the medical device 100 of FIGS. 1 to 5. The clip 150 may be positioned on the outer surface of the mouth of the patient. The clip 150 may be used to control the distance between the inner edges of the suction element 102 and the irrigation element 104 of the medical device 100 once it has been placed within the oral cavity of the patient. Accordingly, the clip 150 may be used to alter the diameter of the mouthpiece to accommodate for variations in anatomy of jaw size of the patient. Further, the width adjustment accommodated for by the clip 150 may ensure that the outer edge of the suction element 102 and the outer edge of the irrigation element 104 are in the appropriate position to be in contact with the inner surface of one or both of the cheeks within the oral cavity of the patient.

In at least one embodiment, the suction port 120 and the irrigation port 126 may each comprise an indentation (e.g., a groove) for placement of the clip 150. The edges of the slots 152 and 156 of the clip 150 may be inserted into these indentations. These indentations may be located at any distance from the suction element 102 and irrigation element 104 of the medical device 100 to allow for appropriate sizing for the patient. In one embodiment, the indentations for receiving the clip 150 may be located closer to the suction element 102 and irrigation element 104. The insertion of the clip 150 into the indentations, in this embodiment, may only increase the distance between the suction element 102 and the irrigation element 104 by a small amount. Alternatively, in at least one embodiment, the indentations for receiving the clip 150 may be located further away from the suction element 102 and the irrigation element 104. The insertion of the clip 150 into the indentation, in such embodiments, may increase the distance between the suction element 102 and the irrigation element 104 by a larger amount. Such embodiments may be used for patients with larger mouths to allow the medical device 100 to be placed as disclosed herein.

In at least one embodiment, the suction port 120 and the irrigation port 126 may not comprise an indentation. In such embodiments, the clip 150 may be positioned snugly on the suction port 120 and the irrigation port 126 while having the ability to slide along the ports. As such, the further from the outer surface of the mouth (i.e., the outer surface of the lips) of the patient the clip 150 is slid, the further apart the suction element 102 and the irrigation element 104 of the medical device 100 are moved relative to one another. In each embodiment disclosed herein, the clip 150 may remain in place on the suction port 120 and irrigation port 126 to ensure the medical device 100 remains in position within the oral cavity of the patient after the medical device 100 has been inserted into the patient's mouth.

In another embodiment, the clip 150 may be placed directly in contact with the outer surface of the mouth (i.e., the lips) of the patient. The clip 150 may remain in position abutting against the outside of the patient's mouth to help seal the mouth as much as possible thereby avoiding or eliminating the possibility of pathogens or aerosols from escaping or entering the patient's mouth in addition to reducing exposure to bodily fluid splashed from the mouth.

Referring now to FIGS. 9A to 9C, shown therein are several embodiments of clips 150a-150c that may be releasably coupled to the suction port 120 and irrigation port 126 of the medical device 100 as shown in FIGS. 1 to 5, or another embodiment of the medical device. In one embodiment, as shown in FIG. 9A, the first slot 152a and third slot 156a are located at an increased distance from one another in relation to the embodiment shown in FIGS. 8A-8C. The clip 150a may, when coupled to the suction port 120 and irrigation port 126 of the medical device 100, decrease the distance between the inner edges of the suction element 102 and the irrigation element 104. The decrease in distance may allow the device 100 to be used for patients with a smaller oral cavity. FIG. 9B shows a second embodiment of the clip 150b where the first slot 152b and third slot 156b are located at an intermediate distance from one another. FIG. 9C shows a third embodiment of the clip 150c wherein the first slot 152c and third slot 156c are located at a decreased distance from one another in relation to the embodiments shown in FIGS. 8A-8B. The clip 150c may, when coupled to the suction port 120 and irrigation port 126 of the medical device 100, increase the distance between the inner edges of the suction element 102 and the irrigation element 104. The increase in distance may allow the device to be used for patients with a larger oral cavity.

Further embodiments (not shown) may be used wherein the first slot 152 and third slot 156 are located at a further distance apart or a smaller distance apart, relative to what is shown in FIGS. 9A-9C, to accommodate patients with differently sized oral cavities.

It should be noted that in the various embodiments of the clips 150-150c described herein that the clips 150-150c may be made of a similar material as the medical devices described herein. Furthermore, the material used to make the clips 150-150c is preferably flexible to allow the outer portions of the clips 150-150c to be contoured such that they wrap around and touch the outer surfaces of the patient's mouth and cheeks to improve the amount of sealing that is provided by the clips 150-150c and the medical devices described herein during use.

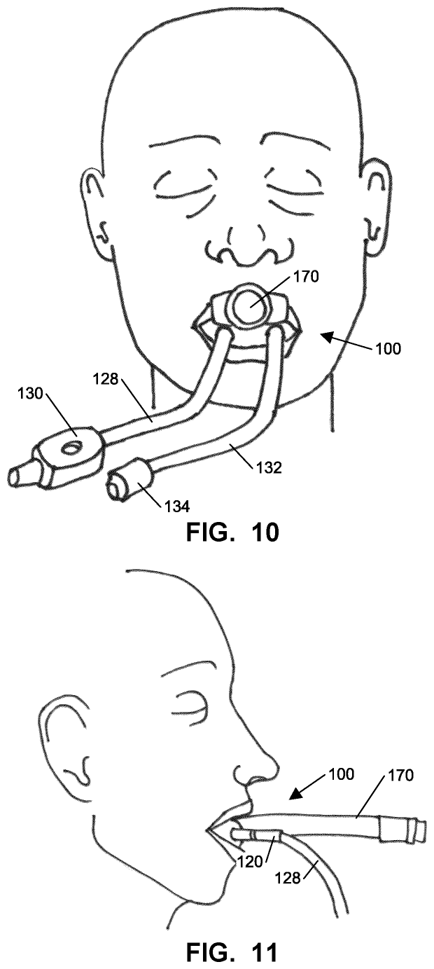

Referring now to FIGS. 10 to 11, shown therein are front and side views of a patient wearing in which one embodiment of the medical device 100 from FIGS. 1-5 has been inserted. As shown, the medical device 100 is placed within the mouth of the patient and an endotracheal tube 170 may be positioned within the mouth of the patient above the medical device 100. The medical device 100 and the endotracheal tube 170 may be releasably coupled to one another. For example, the bridge of the medical device 100 may be shaped to make a releasable friction fit with a portion of the outer perimeter of the endotracheal tube 170. In at least one embodiment, the medical device 100 and the endotracheal tube 170 may be provided together in a kit.

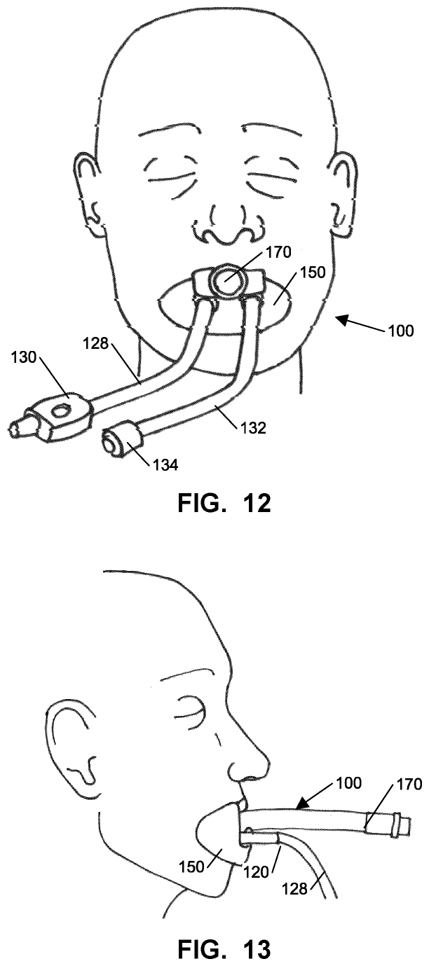

Referring now to FIGS. 12 to 13, shown therein are front and side views of a patient wearing one embodiment the medical device 100 from FIGS. 1-5 combined with the clip 150 of FIGS. 8A-9C. As shown, the clip 150 is positioned outside the mouth of the patient. The endotracheal tube 170 is positioned within the mouth of the patient and positioned above the medical device 100 and also above the second slot 154 of the clip 150. In at least one embodiment, the medical device 100, the clip 150 and the endotracheal tube 170 may be provided together in a kit.

In at least one embodiment, the medical device 100 and/or the clip 150 may be provided with disposable oral care products to form a kit. The disposable oral care products may include an electric toothbrush, non-foaming toothpaste, or any other disposable oral care product.

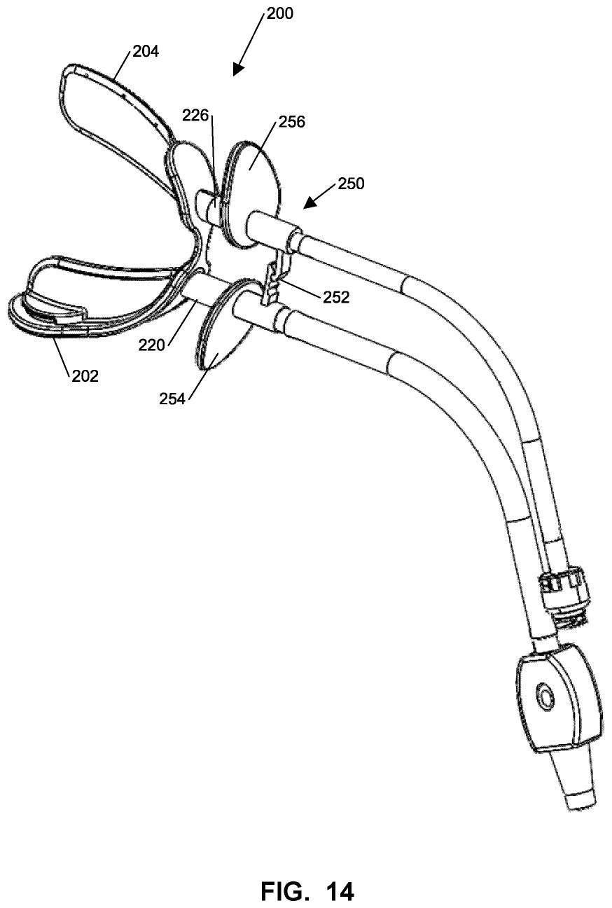

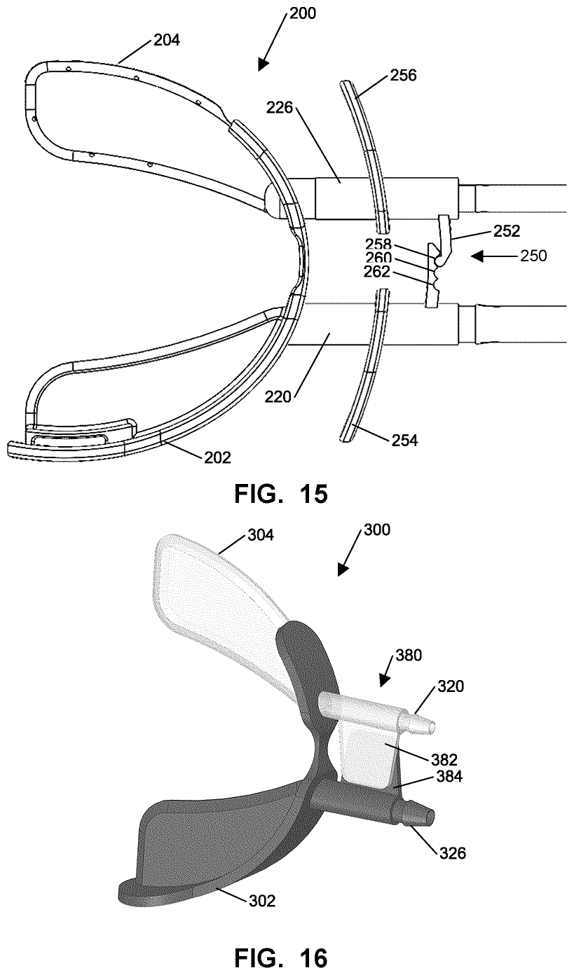

Referring now to FIGS. 14 and 15, shown therein are a top perspective view and a partial top view of another embodiment of a medical device 200 in accordance with the teachings herein. The medical device 200 is similar to medical device 100 but the medical device 200 also includes a locking mechanism 250 instead of the clip 150. Accordingly, the medical device 200 generally operates in a similar manner as was discussed for medical device 100 but with some differences described below. The locking mechanism 250 may be used to alter the distance between the inner edges of the suction element 202 and the irrigation element 204. The locking mechanism 250 includes a ratchet 252. The medical device 200 may also include a first mouthpiece component 254 and a second mouthpiece component 256. One portion (e.g., a first arm) of the ratchet 252 is connected to the suction port 220 and another portion (e.g., a second arm) of the ratchet 252 is connected to the irrigation port 226 of the medical device 200. One portion of the ratchet 252 includes a clip or hook and the other portion of the ratchet 252 includes ridges or teeth that can be releasably engaged by the clip/hook. The ratchet 252 may be adjusted from a first position 258 (as shown in FIG. 15) to a second position 260, a third position 262, or any other position based on the number of ridges or teeth that are included in the ratchet 252. The ratchet 252 may comprise any number of positions for the teeth/ridges to be engaged. Further, the ratchet 252 may include any number of ridges or teeth. The ridges or teeth may be any shape and/or able to lock with any corresponding position of the clip/hook on the ratchet 252. The ratchet 252, when in the first position 258, such that the clip of one arm of the ratchet engages the ridge or tooth that is furthest located on the second arm of the ratchet relative to the connection of the second arm on one of the ports 220 or 226 of the device 200 may increase the distance between the inner edges of the suction element 202 and the irrigation element 204 by a first amount. In contrast, the ratchet 252, when in the third position 262 where the clip engages the closest located ridge/tooth relative to the connection of the second arm to one of the ports 220 or 226 of the device 200, may increase the distance between the inner edges of the suction element 202 and the irrigation element 204 by a second amount that is larger than the first amount.

The first mouthpiece component 254 and the second mouthpiece component 256 may be releasably placed on the outside of the mouth of the patient when the device 200 is in use. The first and second mouthpiece components 254, 256 may provide the user with a way to easily grasp and handle the medical device 200 for appropriate placement into and removal from the mouth of the patient.

In at least one embodiment, the locking mechanism 250 may be releasably connected to the suction port 220 and the irrigation port 226. For example, the arms of the ratchet may include clips or hooks that releasably engage the ports 220 and 226. Alternatively, in at least one embodiment, the locking mechanism 250 may be permanently connected to the suction port 220 and the irrigation port 226.

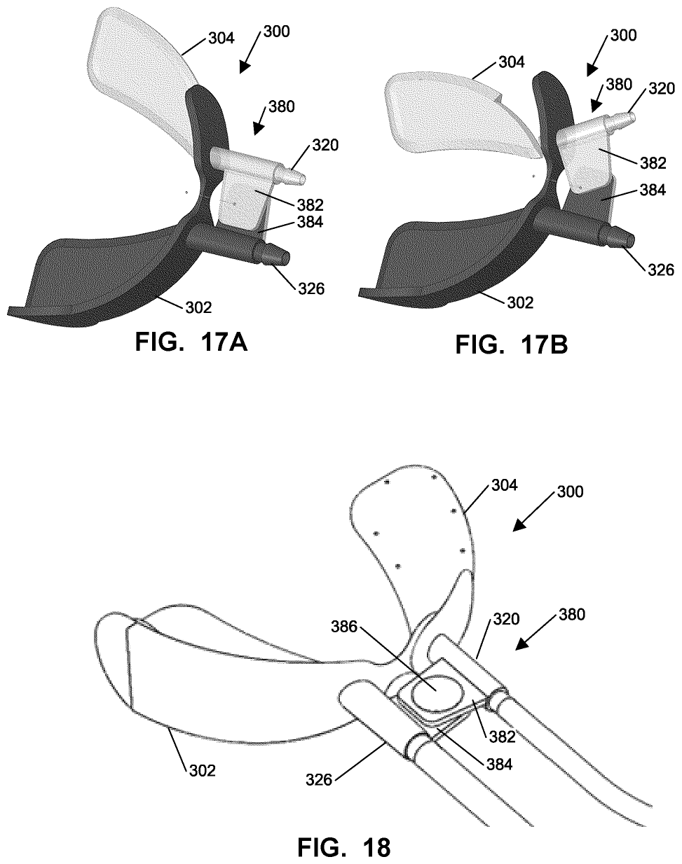

Reference is now made to FIGS. 16, 17A and 17B. FIG. 16 shows a top perspective view of another example embodiment of a medical device 300 in accordance with the teachings herein. FIG. 17A shows a top perspective view of the medical device 300 in a wide locked position. FIG. 17B shows a top perspective view of the medical device 300 in a narrow locked position. The medical device 300 includes the irrigation holes and suction inlet as was discussed for other embodiments described herein although they are not shown in these figures for simplicity of illustration. The medical device 300 generally operates in a similar manner as was discussed for medical device 100 but with some differences described below.

The medical device 300 includes an optional handle assembly 380 that is connected to the suction port 320 and the irrigation port 326. The handle assembly 380 includes a first tab 382 connected to the suction port 320 and a second tab 384 connected to the irrigation port 326. The first and second tabs 382 and 384 may be referred to as male and female connectors, respectively. The first tab 382 and the second tab 384 may be pinched together by the thumb and forefinger of the user. The first tab 382 and the second tab 284 may further be used as a portion of the medical device 300 for the user to hold onto while compressing the mouthpiece during placement within or removal from the oral cavity of the patient.

As shown in FIG. 17A, the inner edges of the suction port 320 and the irrigation port 326 may be moved away from one another by the user such that they are in a compressed position, by pressing on the outer horizontal surfaces of the first tab 382 and the second tab 384. In at least one embodiment, the first tab 382 and the second tab 384 may include elements (not shown) that lock together when the tabs 382 and 284 are compressed together and hold the position of the suction port 320 and the irrigation port 326 in the compressed position where the distance between the inner edges of the suction element 302 and the irrigation element 304 is increased which may be done after the device 300 is inserted into the patient's mouth so that the device 300 can be more firmly fitted into a patient with a larger oral cavity.

As shown in FIG. 17B, the suction port 320 and the irrigation port 326 may be moved apart by a user such that they are in an expanded position. This may be done by a user pressing inserting their finger or another object between the inner horizontal surfaces on the first and second tabs 382 and 384. The first tab 382 and the second tab 384 may then be locked together in embodiments where locking elements are included to hold the position of the suction port 320 and the irrigation port 326 in an expanded position. In such a position, the distance between the suction element 302 and the irrigation element 304 is decreased so as to fit within the oral cavity of a patient when the device 300 is being inserted into the patient's mouth.

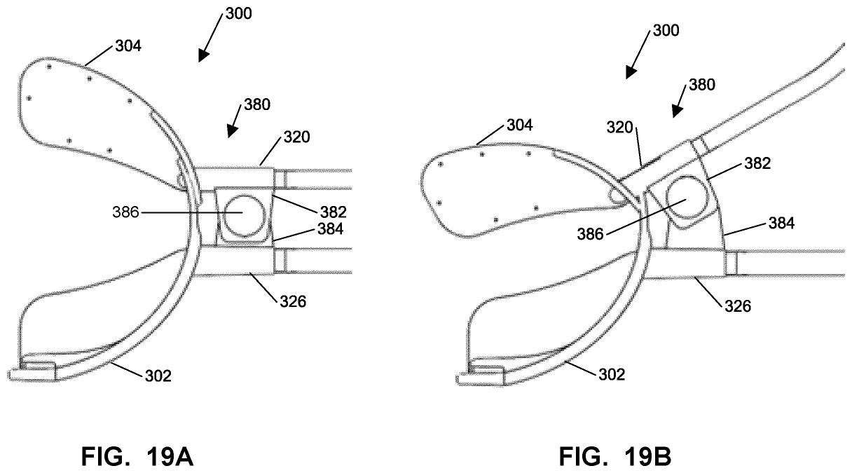

Reference is now made to FIGS. 18, 19A and 19B. FIG. 18 shows a top perspective view of another example embodiment of a medical device 300 in accordance with the teachings herein. FIG. 19A shows a top view of the medical device 300 in a wide locked position. FIG. 19B shows a top view of the medical device 300 in a narrow locked position.

The medical device 300 includes a handle assembly 380 that is connected to the suction port 320 and the irrigation port 326. The handle assembly 380 includes a first tab 382 connected to the suction port 320 and a second tab 384 connected to the irrigation port 326. The handle assembly may further comprise a fastener 386, such as a button, pin or a rivet, for example, on the first tab 382. The second tab 384 of the handle assembly 380 may further comprise one or more slots, holes, or any other receiving component to receive the fastener 386. The first tab 382 and the second tab 384 may be pinched together by the thumb and forefinger of the user. The fastener 386 may be pushed down by the user to connect with one of the receiving components of second tab 384 to lock the handle assembly 380 in position. The first tab 382 and the second tab 284 may further be used as a place for the user to hold while compressing the mouthpiece during placement within the oral cavity of the patient. In other embodiments, the positions of the tabs 382 and 384 may be reversed so that they are connected to the suction and irrigation ports, respectively, or other portions of the medical device 300.

As shown in FIG. 19A, the suction port 320 and the irrigation port 326 may be moved together by the user such that they are in a compressed position, by pressing on the bottoms of the first tab 382 and the second tab 384. The fastener 386 may lock into one of the receiving components when compressed together and hold the position of the suction port 320 and the irrigation port 326 in the compressed position where the distance between the inner edges of the suction element 302 and the irrigation element 304 is increased which may be done after the device 300 is inserted into the patient's mouth so that the device 300 can be more firmly fitted into a patient with a larger oral cavity.

As shown in FIG. 19B, the suction port 320 and the irrigation port 326 may be pulled apart by a user such that they are in an expanded position. This may be done by a user pressing down on the first and second tabs 382 and 384 when they are not locked with one another. The fastener 386 may lock into one of the receiving components when pushed downwards to hold the position of the suction port 320 and the irrigation port 326 in an expanded position. This may occur when there are multiple receiving components or multiple tabs that are each laterally spaced from one another thereby allowing the distance of the ports 320 and 326 to be locked at different distances with respect to one another. In the expanded position, the distance between the inner edges of the suction element 302 and the irrigation element 304 is decreased so as to fit within the oral cavity of a patient when the device 300 is being inserted into or removed from the patient's mouth.

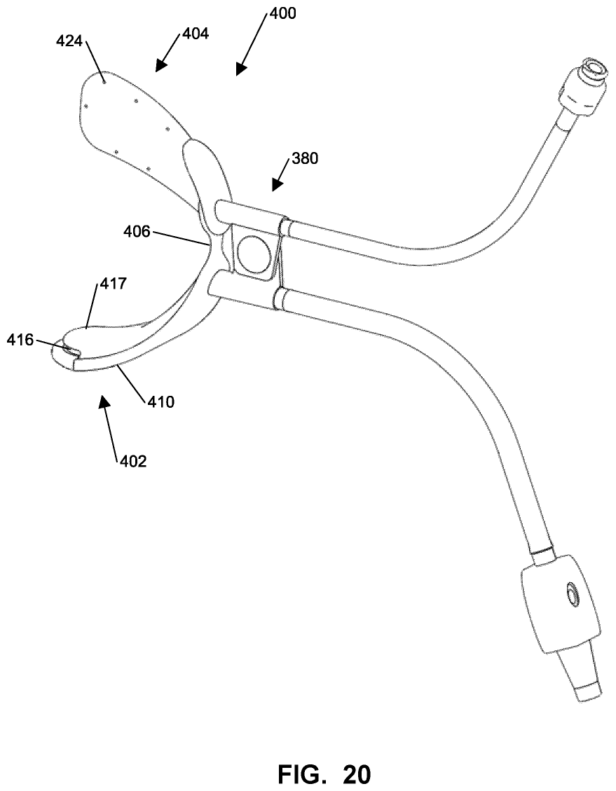

Referring now to FIG. 20, shown is a perspective view of another example embodiment of a medical device 400 in accordance with the teachings herein. The medical device 400 comprises a suction element 402 and an irrigation element 404. The properties, characteristics and functions that were described for the suction and irrigation elements 102, 104 of medical device 100 may generally apply to the suction and irrigation elements 102, 404. Accordingly, the medical device 400 generally operates in a similar manner as was discussed for medical device 100 but with some differences described below. The suction element 402 may comprise a paddle 410 and at least one suction inlet 420. The suction element 402 does not include a plate (which also may be called a bite plate or a bite wing). The paddle 410 may comprise an elongated arm that is of a uniform construction (i.e., approximately the same height the entire length of the arm). In alternative embodiments, the paddle 410 may comprise an arm wherein the distal end of the paddle 410 is enlarged and the arm decreases in height as it gets closer to the bridge 406, forming a shape similar to that of a teardrop (as shown in FIG. 21B). The paddle 410 may be placed on the outer side of the upper and lower teeth of the patient such that the paddle 410 is located between the outer surface of the patient's upper and lower teeth and the inner surface of the cheek of the patient's oral cavity.

Referring now to FIGS. 21A and 21B, shown therein are partial views of example embodiments of the suction element 402 of the medical device 400 of FIG. 20. FIG. 21A shows a top view of medical device 400. FIG. 21B shows a cross-sectional right side view of the suction element 402 of medical device 400.

The suction element 402 comprises at least one suction inlet 416 for suctioning fluid out of the oral cavity. The paddle 410 may have a raised surface 417 on the inner surface. The suction inlet 416 of the suction element 402 may be defined by an opening formed on a distal portion of the raised surface 417. In alternative embodiments, there may be more than one suction inlet 416 that is included in the suction element 402 or the suction inlet may be formed on another portion of the raised surface 417. In alternative embodiments, the paddle 410 may not have a raised surface 417 and/or the one or more suction inlets 416 included on the suction element 402 may be formed on any portion of the paddle 410. Alternatively, in at least one embodiment, as shown in FIG. 21B, there may be multiple suction inlets 416 near the distal end of the paddle 410. Further shown in FIG. 21B is the direction of fluid flow into the suction inlets 416 (as shown by the arrows). A line A-A represents the tissue of the inner cheek of the oral cavity and is shown in FIG. 21B. As can be seen, the suction inlets 416 are a slight distance from the edge of the paddle 410. As such, intake of the fluid through the suction inlets 416 is set back from the edge of the paddle 410 that contacts the mucosal tissue, thereby minimizing chances of occlusion.

Referring now to FIG. 21C, shown therein is a top view of a horizontal cross section of the medical device 400 of FIG. 20 without showing the irrigation or suction lines that are external to the irrigation and suction ports, respectively. The medical device 400, as well as the other embodiments of the medical devices described herein as well as alternatives thereof, includes an interior irrigation channel (here comprised of irrigation subchannels 404a and 404b) that is located within the irrigation element 404 and has a first end that is fluidly coupled to an irrigation inlet port 404i and a second end that is fluidically coupled to one or more irrigation outlets 424 (only one of which is labelled for simplicity). The medical device 400, as well as the other embodiments of the medical devices described herein as well as alternatives thereof, also includes an interior suction channel (here comprised of irrigation subchannels 402a and 402b) within the suction element 402 has a first end that is fluidly to a suction outlet port 4020 and a second end that is fluidically coupled to at least one suction inlet 416. The interior irrigation and suction channels are not connected to one another.

The interior irrigation and suction channels may have varying shapes and are not connected to one another. For example, the suction channel may have a first cross-section closer to the suction inlet 416 and a second cross-section closer to the suction outlet port 4020 where the first cross-section may be larger than the second cross-section in at least one embodiment. Alternatively, the first cross-section may be smaller than the first cross-section in some cases. For the interior irrigation channel, the portion 404b may irregularly shaped such that there are curved portions near the irrigation outlets to aid in directing the irrigation fluids to the irrigation outlets as shown. Alternatively, the interior irrigation channel may have smooth walls without these curved portions in some cases. In other embodiments, the interior irrigation and suction channels may have different shapes and sizes.

The remaining features of the medical device 400, may be of the same properties, characteristics and functions of the previous medical devices that were described. For example, the medical device 400 may, as in the illustrated embodiment, comprise a handle feature 380 as described in relation to FIGS. 16 and 17A-17B. In an alternative embodiment, medical device 400 may comprise a locking mechanism as disclosed in relation to FIGS. 14 and 15. In an alternative embodiment, medical device 400 may, instead of the locking mechanisms of FIGS. 14 to 17B, comprise a clip as disclosed in relation to FIGS. 8A-9C.

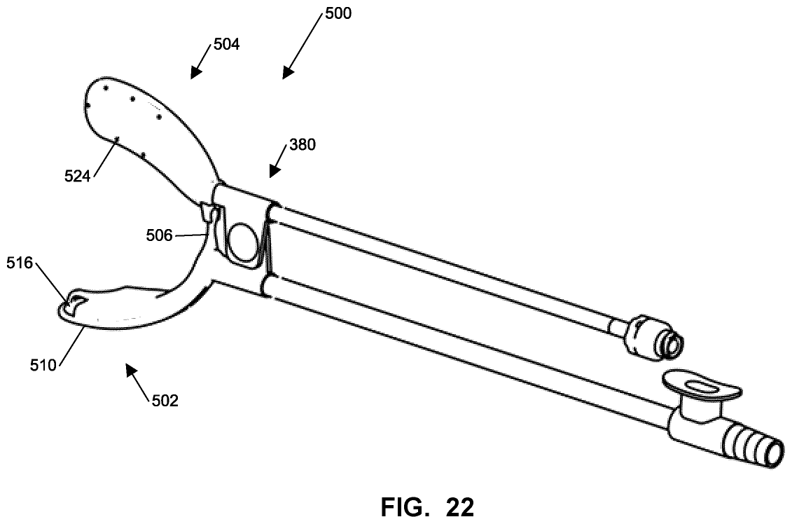

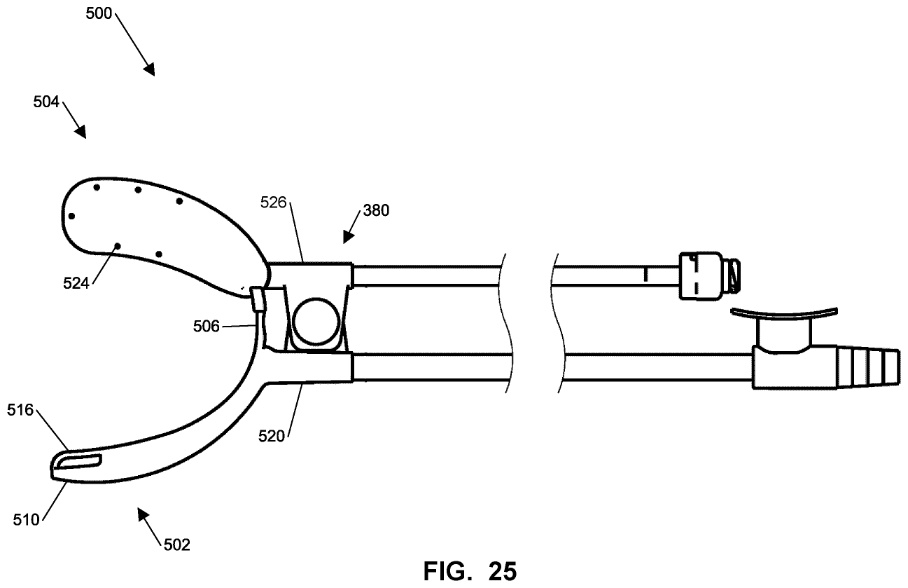

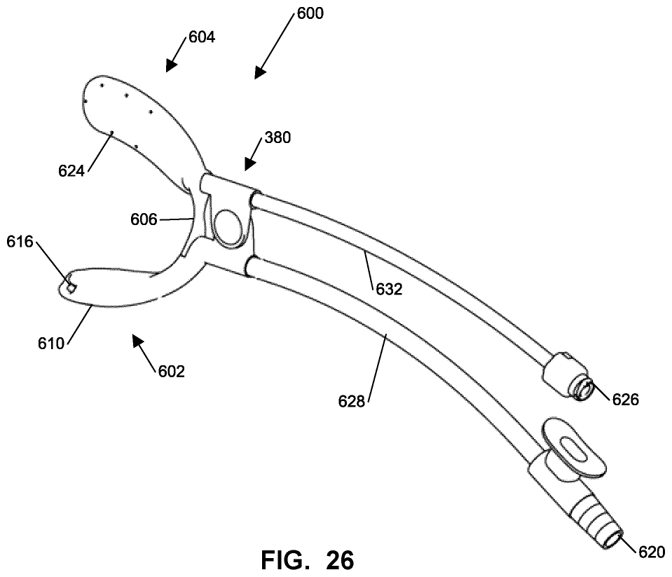

Referring now to FIGS. 22 and 25, shown therein is a perspective view of another example embodiment of a medical device 500 in accordance with the teachings herein. The medical device 500 comprises a suction element 502 and an irrigation element 504. The properties, characteristics and functions that were described for the suction and irrigation elements 102, 104 of medical device 100 may apply to the suction and irrigation elements 502, 504. Accordingly, the medical device 500 generally operates in a similar manner as was discussed for medical device 100 but with some differences described below. However, suction element 502 and irrigation element 504 has also both been modified in certain aspects in comparison to suction element 402 and irrigation element 104, respectively, as will now be described.

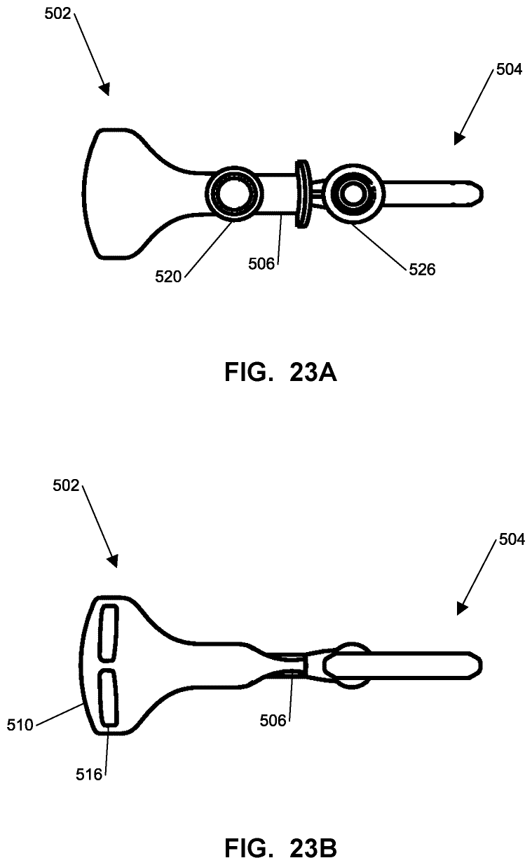

Referring now to FIGS. 23A and 23B, shown therein are front and back views of the medical device 500. FIG. 23A shows a front view of the medical device 500, and in particular, an example of the location of the suction port 520, the irrigation port 526, and the shape of the bridge 506. FIG. 23B shows a back view of the suction element 502 and the irrigation element 504 with the suction ports 516 being shown on the paddle 510 of the suction element 502.

The bridge 506 of the medical device 500 connecting the suction element 502 and the irrigation element 504 may be comprised of a relatively flat section that is has flat upper and lower surfaces and is thinner (e.g., lower or smaller) in profile relative to the bridge of the previous medical device embodiments described herein. The top edge of the bridge 506 may be low and flat relative to the upper edge of the suction element 502 to allow for, in at least one embodiment, receiving an endotracheal tube to be located there unimpeded by the medical device 500 regardless of the lateral position of the endotracheal tube within the patient's mouth. The endotracheal tube may be moved laterally within the mouth of the patient across the top of the bridge 506, to a greater degree than with the previous medical device embodiments herein, thereby allowing medical professionals to move the location of the endotracheal tube without the bridge 506 of the medical device 500 being in the way. The bridge 506 having a thin height may allow, in at least one embodiment, for the medical device 500 to be flipped over within the oral cavity of the patient to allow for suctioning on either side of the mouth. As such, the profile of the bridge 506 may remain low and flat relative to the suction element 502 so that the endotracheal tube may be placed and moved appropriately no matter the orientation of the medical device 500 within the patient's mouth (i.e., regardless of whether the medical device 500 has the orientation shown in FIGS. 23A and 23B or is rotated 180 degrees relative to the orientation shown in FIGS. 23A and 23B).

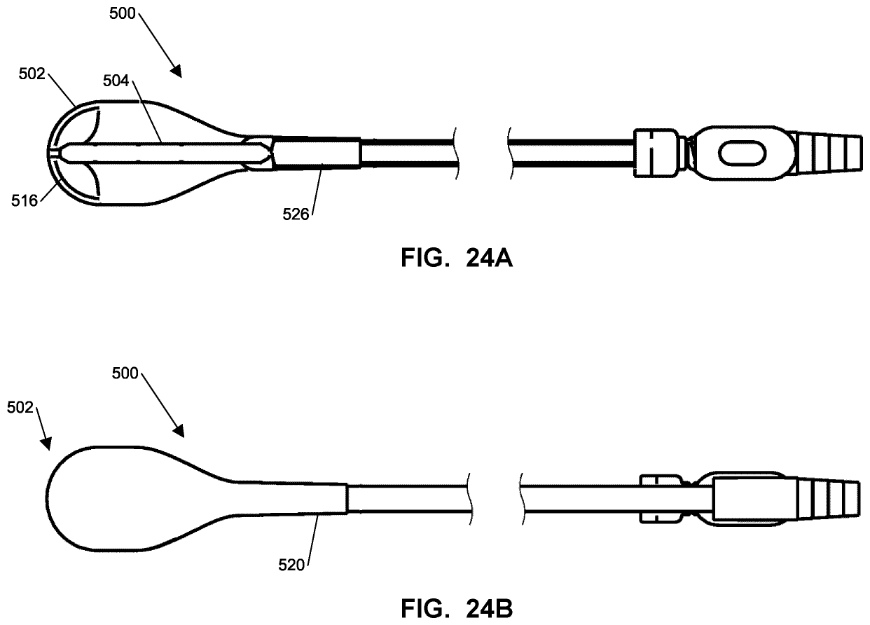

Referring now to FIGS. 24A and 24B, shown therein are right and left side views of the medical device 500. In particular, FIG. 24A shows the irrigation and suction elements 502 and 504, while FIG. 24B shows an outer side of the suction element 502.

Suction element 502 is curved and smooth along all edges to eliminate any sharp components. This allows for the medical device 500 to prevent injury or damage when in contact with the mucous membranes of a patient's mouth. For example, the smooth edges of the suction element 502 reduce the likelihood of tissue damage during insertion into and removal from the mouth of the patient.

Suction element 502 of medical device 500 may comprise at least one suction inlet 516 for suctioning fluid out of the oral cavity. The suction inlet 516 of the suction element 502 may be defined by an opening formed on the inner side of the suction element 502, in a similar location as shown in FIG. 24A. Suction inlet 516 may have an elliptical, semi-circular or almond-shaped opening. In at least one embodiment, the suction inlet 516 may be any shape with a smooth curvature. In at least one embodiment, as shown in FIG. 24A for example, there may be two suction inlets 516. As can be seen, the suction inlets 516 are a slight distance from the edge of the paddle 510 that extends rearmost into the patient's mouth during use. As such, intake of the fluid through the suction inlets 516 is set back from the edge of the paddle 510 that contacts the mucosal tissue, thereby minimizing chances of occlusion. In at least one embodiment, the suction inlet 516 may be located at other locations on the paddle 510 of the suction element 502. In at least one embodiment, the suction inlet 516 may be sized so as to reach from the upper edge of the suction element 502 to the bottom edge of the suction element 502 along the inner side. In another alternative embodiment, there may be more than two suction inlets that may be located at additional locations on the paddle 510 of the suction element 502 such as closer to the middle and/or front of the patient's mouth while including the rearmost position shown in FIG. 24A.

The remaining features of the medical device 500, may be of the same properties, characteristics and functions of other medical devices described herein. For example, the medical device 500 may, as in the illustrated embodiment, comprise a handle feature 380 as described in relation to FIGS. 16 and 17A-17B. In an alternative embodiment, medical device 500 may comprise a locking mechanism as disclosed in relation to FIGS. 14 and 15. In an alternative embodiment, medical device 500 may, instead of the locking mechanisms of FIGS. 14 to 17B, comprise a clip as disclosed in relation to FIGS. 8A-9C.

Referring now to FIG. 26, shown therein is a perspective view of another example embodiment of a medical device 600 in accordance with the teachings herein. The medical device 600 comprises a suction element 602 and an irrigation element 604. The properties, characteristics and functions that were described for the suction element 502 and the irrigation element 504 of medical device 500 may generally apply to the suction element 602 and the irrigation element 604 of medical device 600, respectively. However, suction element 602 has been modified in certain aspects in comparison to suction element 502. Accordingly, the medical device 500 generally operates in a similar manner as was discussed for medical device 100 but with some differences. which will now be described.

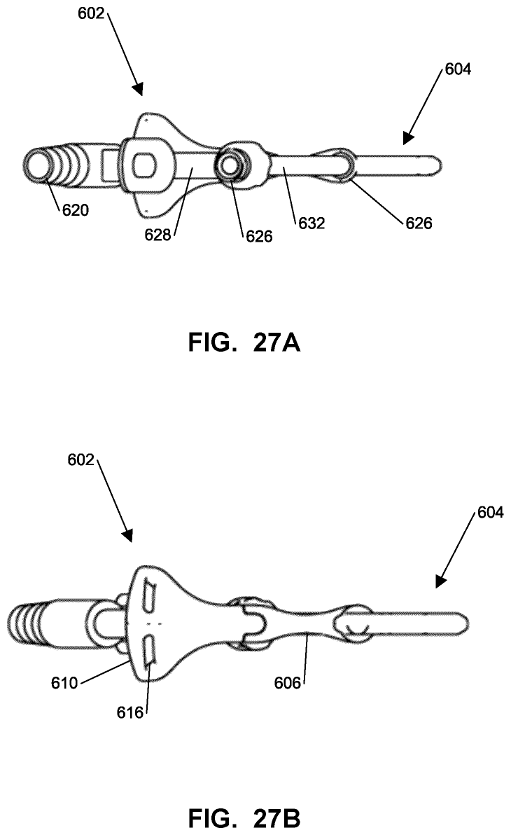

Referring now to FIGS. 27A and 27B, shown therein are front and back perspective views of the medical device 600. FIG. 27A shows a front perspective view of the medical device 600, and in particular, an example of the location of the suction port 620, irrigation port 626, suction line 628 and irrigation line 632. FIG. 27B shows a back view of the suction element 602 and the irrigation element 604 with the suction inlets 616 being shown on the paddle 610 of the suction element 602, as well as the shape of the bridge 606.

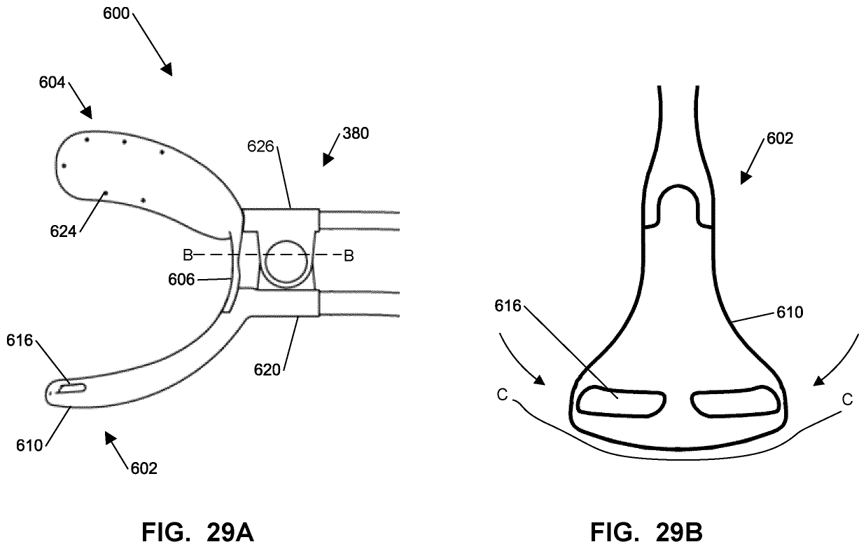

The bridge 606 of the medical device 600 connecting the suction element 602 and the irrigation element 604 may be comprised of a relatively flat section that has flat upper and lower surfaces and a height such that it has a lower profile similar to that described for the bridge 506 of medical device 500. For example, the height of the bridge 606 may be substantially similar or about the same as the height of the irrigation element 604. Referring now to FIG. 29A, the bridge 606 may have an increased thickness along axis B-B relative to the bridge of the previous medical device embodiments described herein. This increased thickness along axis B-B may create a product that is less prone to breakage during use, as well as to increase ease of manufacture. For example, increasing the front to back thickness of the bridge 606 may allow, in at least one embodiment, the bridge 606 to be manufactured more easily as the product has more body, while maintaining the benefits from the lower profile similar to bridge 506 of medical device 500 (i.e., flipped over within the oral cavity, ability to move the endotracheal tube easily, etc.).

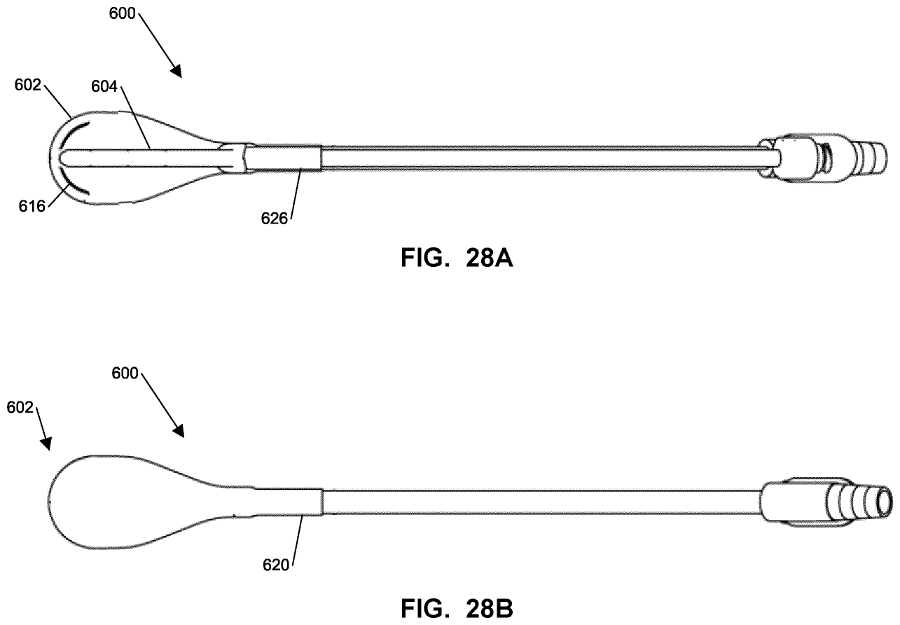

Referring now to FIGS. 28A and 28B, shown therein are right and left side views of the medical device 600. In particular, FIG. 28A shows the irrigation and suction elements 602 (inner surface) and 604, while FIG. 28B shows an outer side of the suction element 602. Suction element 602 of medical device 600 may comprise at least one suction inlet 616 for suctioning fluid out of the oral cavity. The suction inlet 616 of the suction element 602 may be defined by an opening formed on the inner side of the suction element 602, in a somewhat similar location as shown in FIG. 24A. However, suction inlet 616 may have a curved thin oval shaped opening with upper and lower arcs that are both curved upwardly and sized such that the openings appear more like a slit when viewed from the right side, such as in FIG. 28A. In such embodiments, the raised profile of suction element 602 creates a depth to the suction inlet 616 to allow for adequate fluid flow through suction inlet 616. The shape of the opening of suction inlet 616 may remove any corners or sharp edges that may be shown in previous embodiments to reduce patient discomfort when the medical device 600 is placed in the mouth. In at least one embodiment, as shown in FIG. 28A, there may be two suction inlets 616, for example. In at least one embodiment, the suction inlet 616 may be located at other locations on the paddle 610 of the suction element 602 or have different sizes. For example, in at least one embodiment, the suction inlet 616 may be sized so as to reach from near the upper edge of the suction element 602 to near the bottom edge of the suction element 602 along the inner surface (e.g., there may be one inlet that is shaped as a slit and may be a combination of upper and lower inlets 616). In another alternative embodiment, there may be two or more upper and lower suction inlets that are located adjacent and to the right of suction inlets 616 in FIG. 28A.

Referring now to FIGS. 29A and 29B, shown therein are partial views of another example embodiment of the suction element 602 of the medical device 600 of FIG. 26. FIG. 29A shows a top view of medical device 600. FIG. 29B shows a cross-sectional right side view of the suction element 602 and suction inlet 616 of medical device 600. The suction inlet 616 of the suction element 602 may be defined by an opening formed on a distal portion of paddle 610. The suction inlet 616 may have a rounded rectangular shape with upper and lower portions that are curved upwards and downwards, respectively and the vertical extent of the slits may be closer to the vertical edges of the paddle 610. The angle of FIG. 29B shows the depth of the suction inlet 616 along the profile of the suction element 602. In alternative embodiments, there may be two suction inlets 616 (as shown) or more than two suction inlets 616 that are included in the suction element 602. The one or more suction inlets 616 included on the suction element 602 may be formed on other portions of the paddle 610 as was described previously in other embodiments. Further shown in FIG. 29B is the direction of fluid flow into the suction inlets 616 (as shown by the arrows). A line C-C represents the tissue of the inner cheek of the oral cavity and is shown in FIG. 29B when the device 600 is in use. As can be seen, the suction inlets 616 are a slight distance from the upper and lower edges of the paddle 610 and set inwards from the distal end of the suction element 602. For example, the distance of the suction inlet 616 from the distal end of the suction element 602 may be substantially similar or the same as the width of the suction inlet 616. As such, intake of the fluid through the suction inlets 616 is set back from the edge of the paddle 610 that contacts the mucosal tissue, thereby minimizing chances of occlusion.

The remaining features of the medical device 600, may be of the same properties, characteristics and functions as other medical device embodiments described herein. For example, the medical device 600 may, as in the illustrated embodiment, comprise a handle feature 380 as described in relation to FIGS. 16 and 17A-17B. In an alternative embodiment, medical device 600 may comprise a locking mechanism as disclosed in relation to FIGS. 14 and 15. In an alternative embodiment, medical device 600 may, instead of the locking mechanisms of FIGS. 14 to 17B, comprise a clip as disclosed in relation to FIGS. 8A-9C.

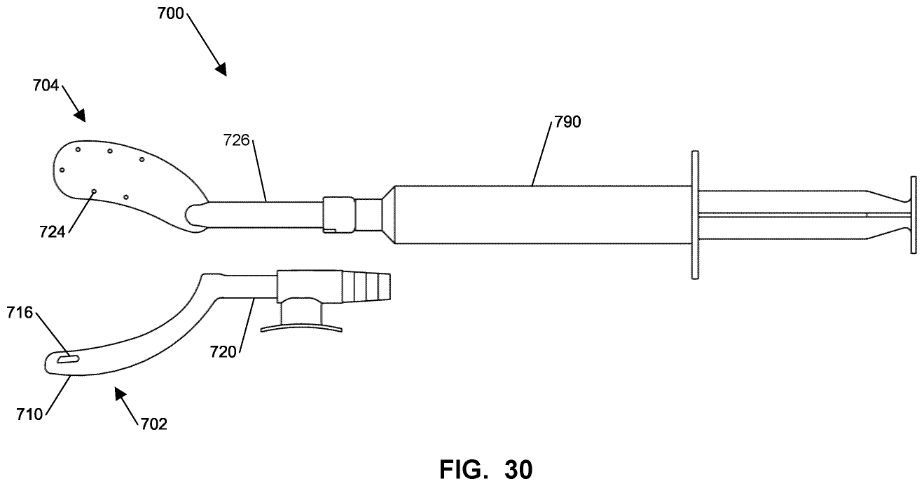

Referring now to FIG. 30, shown therein is a top view of another example embodiment of a medical device 700 in accordance with the teachings therein. The medical device 700 comprises a suction element 702 and an irrigation element 704. The properties, characteristics and functions that were described for the suction element 602 and the irrigation element 604 of medical device 600 may generally apply to suction element 702 and the irrigation element 704, respectively, of the medical device 600. Accordingly, the medical device 700 generally operates in a similar manner as was discussed for medical device 600. However, medical device 700 has been modified in certain aspects in comparison to medical device 600, as will now be described.

The suction element 702 and the irrigation element 704 are separate pieces of the medical device 700. Therefore, medical device 700 may be used so that a single piece at a time to be placed within the mouth of the patient. For example, irrigation element 704 may be placed within the mouth of the patient to irrigate the oral cavity, and then removed. Suction element 702 may then be placed within the oral cavity to suction any fluid, and then removed. In another example, both the irrigation element 704 and the suction element 702 may be placed within the oral cavity simultaneously. Where both the irrigation element 704 and the suction element 702 are placed within the oral cavity together, the user may be required to hold onto these pieces of the medical device 700 to keep them in the appropriate place within the mouth of the patient during use. The two part design of the medical device 700 may be applied to other embodiments of the medical device described herein.

Suction port 720 and irrigation port 726 may be of any length. For example, as shown in FIG. 30, suction port 720 and irrigation port 726 are of a shorter length compared to previous embodiments of the medical devices described herein. However, in an alternative embodiment, suction port 720 and irrigation port 726 may be of an increased length to allow for improved maneuverability by the user. In another alternative embodiment, the suction port 720 and irrigation port 726 may be connected to a wand of any length, to any device for irrigation and/or suctioning of an oral cavity. In at least one embodiment, the irrigation port 726 may be connected to a syringe 790 that is filled with fluid and the plunger of the syringe 790 may be pushed during use to irrigate the patient's mouth. The same syringe 790, when empty, or another empty syringe may be used connected to the suction port to suction the fluid from the patient's mouth.

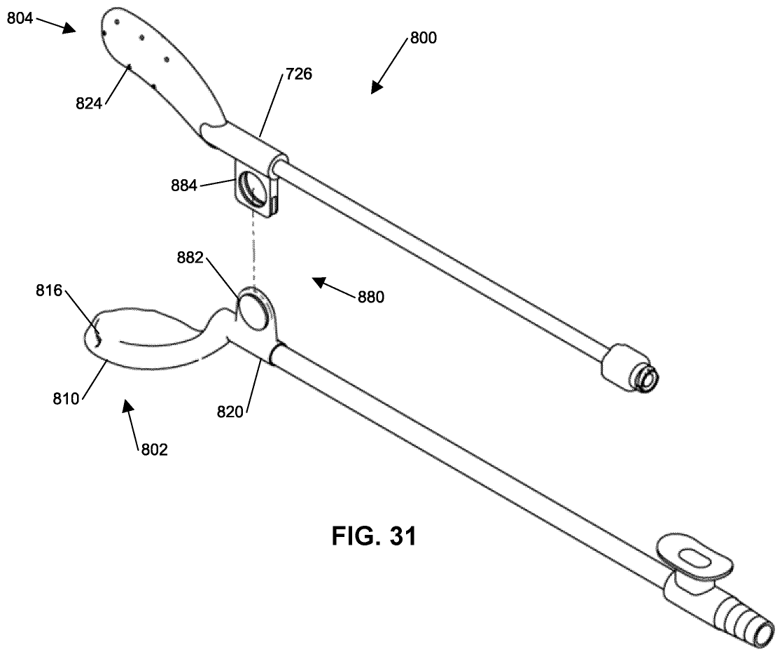

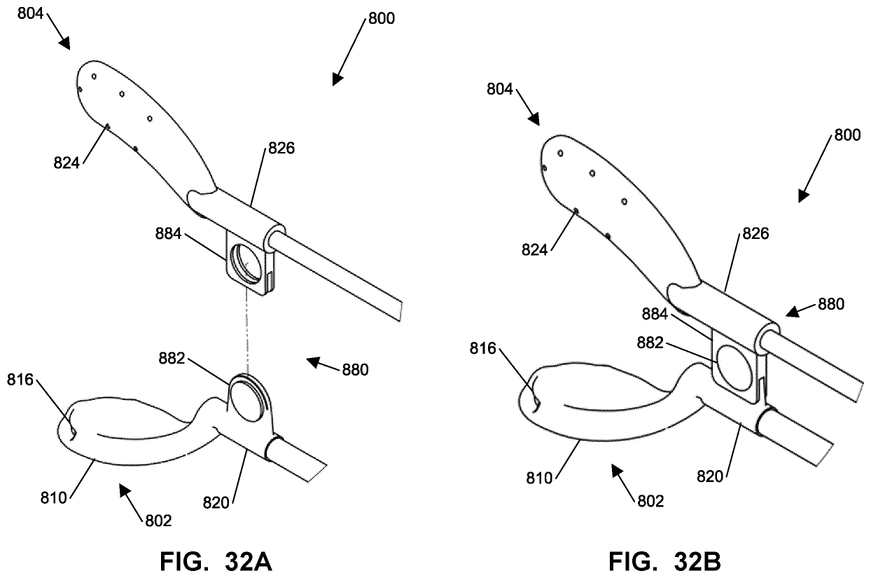

Referring now to FIG. 31, shown therein is a perspective view of another embodiment of a medical device 800 in accordance with the teachings herein. The medical device 800 is somewhat similar to medical devices 600 and 700 in that the medical device 800 may be maintained as two separate elements. The medical device 700 generally operates in a similar manner as was discussed for medical device 600 but with some differences. For example, the medical device 800 also includes a handle assembly 880 that may be used to releasably connect the suction element 802 and the irrigation element 804 together.

The handle assembly 880 of medical device 800 may include a male connector 882 connected to the suction port 820 and a female connector 884 connected to the irrigation port 826. The male connector 882 includes a slight protrusion in the form of a low profile post, pin or boss, while the female connector 884 includes an aperture and a slot that are shaped and sized to releasably receive the post and a tab or flat portion of the male connector 882. In an alternative embodiment, the location of the male and female connectors 882 and 824 may be on the irrigation and suction ports 826 and 820, respectively. The two halves of the medical device 800 may be connected by inserting the post of the male connector 882 into the aperture of the female connector 884, as shown in FIG. 31. The male and female connectors 882 and 884 may be used to connect the suction element 802 and the irrigation element 804 together to keep the position of the medical device 800 at somewhat or fully fixed within the oral cavity of the patient. The handle assembly 880 may further be used as a physical element for the user to hold onto while configuring the mouthpiece during placement within the oral cavity of the patient.

In an alternative embodiment, the male and female connectors 882 and 884 may be implemented such that they are vertically pinched together by the thumb and forefinger of the user to connect and lock the suction element 802 and the irrigation element 804. In another alternative embodiment, the male and female connectors 882 and 884 may connect to one another through use of a ratchet, such as ratchet 252 as shown in relation to FIGS. 14 and 15. In another alternative embodiment, the male and female connectors 882 and 884 may be connected and locked through use of a button snap, a buckle, Velcro, double sided adhesive, screws, pins, or any other mechanism to releasably connect and lock two components together.

A method for irrigation of an oral cavity of a patient is now described. The method includes placing one of the medical devices described herein, such as any of medical devices 100-800 or alternatives thereof, within the oral cavity of the patient, irrigating the oral cavity with fluid delivered using the irrigation element 104-804 and suctioning the fluid out of the oral cavity by suctioning provided through the suction element 102-802. The distal ends of the suction line 128 and irrigation line 132 of the medical device 100-800 may be connected to a wall mounted suction device, an empty syringe or another suction device, and a syringe filled with fluid or an irrigation device, respectively. The irrigation fluid is then provided through the irrigation outlets and fluid in the oral cavity may be suctioned at the same time. The provision of irrigation fluid and suctioning may be done continuously for a certain time duration, or there may be a lag time between the beginning of applying irrigation and the beginning of applying suctioning. Either technique may also be repeated and/or applied periodically over a certain period of time.

In at least one embodiment, the patient may be positioned so that the head and oral cavity of the patient is at a lower position in relation to the feet of the patient (i.e., Trendelenburg position). This patient position may mitigate the risk of fluid being translocated from the oral cavity further down the aerodigestive tract. The patient may then be positioned on their side so that the suction element 102-802 is disposed within the buccal pocket located in the vertically lowest position of the oral cavity of the patient.

The medical devices described herein may be used to irrigate the oral cavity and suction out the irrigation fluids, thereby cleaning and disinfecting a patient's oral cavity. The fluid may be water, saline, or any other fluid such antiseptics, antibiotics or lubricants, or fluid of any kind. Additional cleaning steps such as brushing the teeth or oral cavity with a brush or any of the other cleaning steps previously described above may also be used in conjunction with one of the medical devices described herein or alternative thereof, such as prior to using such devices.

In an alternative embodiment, a method may be applied that further involves nasal irrigation in conjunction with use of the medical device. In such a procedure, fluid may be instilled into the nostril of a patient either at the same time as oral irrigation or separately while suctioning is being performed.

Furthermore, while embodiments of the medical devices described and depicted herein are for use with ventilated patients, alternatives are possible. For example, the medical devices described herein or any alternatives thereof may be used within the dentistry industry for assistance with irrigation and suctioning during certain dentistry procedures. In such applications, the portion of the medical device that is used to accommodate an endotracheal tube may instead be used for placement of certain dental tools for performance of certain dental procedures. In another alternative, the medical devices described and depicted herein or any alternative thereof may be used within any hospital setting wherein the patient is dependent on a staff member and/or other person for oral care.

In addition, any of the suctioning steps may be performed using high pressure suctioning (e.g., a high negative pressure) which may involve using up to about 250 mm Hg or higher such as up to about 400 mm Hg, for example, of suction applied without causing any tissue within the patient's mouth to be drawn into suction inlets and causing obstruction.

In addition, any of the irrigation steps may be performed using low pressure irrigation, intermediate pressure irrigation or high pressure irrigation. High pressure irrigation may use about 250 mL of fluid per minute or greater as is appropriate based on the amount of cleaning that needs to be done and the amount of suction that is being used.

In addition to helping maintain oral hygiene, the various embodiments of the devices and methods descried in accordance with the teachings herein may provide additional protection against other pathogens such as the COVID 19 virus by preventing or minimizing generation of aerosols which may contain the virus or other pathogens. Use of the clip and irrigation/lavage and sanitization procedures described herein help avoid open airway suctioning as well as minimize or prevent aerosolization of pathogens. This is because any leakage from the patient's mouth is reduced or eliminated by use of the clip so that the device more firmly engages the patient's oral cavity. Also, the increased irrigation/lavage and sanitization of the oral cavity that is possible with the devices described herein can be used to reduce aerosols and provide additional protection to front line critical care staff.

In accordance with another aspect of the teachings herein, any of the medical devices described herein or alternatives thereof may be used to obtain oral samples from the patient during use. These oral samples may then be analyzed using known analytical techniques to detect a variety of pathogens, microbes and/or bacteria such as, but not limited to, various fungi or viruses, for example, that may be responsible for various illnesses or negative physiological conditions. These oral samples may be obtained by using the suction element of the various medical devices described herein. In some cases, the sampling may be done without irrigation. In other cases, the sampling may be done after irrigation or at the same time as irrigation. In such cases, the irrigation may help to dislodge certain bacteria and/or pathogens such that they are easier to obtain in the oral sample(s). In either case, since the suction inlets of the various medical devices described herein reach further back into the oral cavity, these devices enable the provision of oral sample(s) further rearward areas of the oral cavity where it might be particularly hard to otherwise obtain such samples. Another benefit is that sampling these difficult to reach areas may allow for the detection and/or quantification of certain bacteria or pathogens that reside there which would otherwise be difficult to sample using known techniques. This may also allow for identifying and quantifying bacteria, fungi, and viruses, for example, that are known to cause illness not limited to pneumonia and systemic illness, for example.

For example, the suction line/port of one of the medical devices described herein or an alternative thereof, might be connected to a suction device for withdrawing fluid and small particulate matter as one or more samples from a patient's oral cavity. The suction device is then activated or engaged with or without irrigating the oral cavity (as described earlier) to provide suction to withdraw the oral sample(s) from the patient's mouth. For example, the suction device might be a syringe where the sample is obtained and may reside in the reservoir of the syringe or a conduit of the syringe that would connect the reservoir to an opening at an end of the syringe that is connected to the suction line/suction port and is opposite the end of the syringe where the plunger is located. The plunger of the syringe may be pulled back away from the body of the syringe when obtaining the sample and then pushed into the body of the syringe when dispending the sample into a sample container or on a petri dish, for example.

Alternatively, a sample container may be used that includes two tubes, which may be referred to as a sample tube and a suction tube for example, and the suction device may be coupled to the suction tube and the sample tube can be coupled to the suction line/suction port of one of the medical devices described herein. The suction device may then be activated/engaged such that one or more oral samples are suctioned out of the patient's oral cavity due to the suction force that is provided by the suction device then through the fluidic coupling to the suction tube, the interior of the sample container, the sample tube, the suction line to the suction element and suction inlets of the medical device. Once the oral sample(s) is visibly seen to be exiting the sample tube and entering into the sample container, the suction device may be deactivated/disengaged to retain the oral sample(s) within the sample container. In alternative embodiments, one of the medical devices described herein or an alternative thereof may be used in a similar manner with other types of sample containers to obtain at least one oral sample.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A medical device for suction and irrigation of an oral cavity in a patient, the device comprising:
a suction element to place at a location of the oral cavity, the suction element comprising an elongated arm and at least one suction inlet to suction fluid out of the oral cavity;
an irrigation element to dispose at a second location of the oral cavity, the irrigation element comprising a plate and one or more irrigation outlets to irrigate the oral cavity with fluid;
a suction port to couple with the suction element for removal of fluid from at least a first portion of the oral cavity; and
an irrigation port coupled with the irrigation element to provide the fluid thereto to irrigate at least a second portion of the oral cavity.

2. (canceled)

3. The device of claim 1, comprising:
a bridge to couple the suction element with the irrigation element.

4.-5. (canceled)

6. The device of claim 3, wherein a center portion of the bridge has a low profile relative to outer portions of the bridge.

7. (canceled)

8. The device of claim 1, comprising:
a bridge; and
the suction port and the irrigation port located at the bridge and laterally offset from a center the bridge and configured to extend outside of the oral cavity during use.

9. The device of claim 1, comprising:
the suction element including a paddle, the paddle including the elongated arm with an enlarged head.

10. The device of claim 1, comprising:
the suction element including a paddle, the paddle including the elongated arm.

11. The device of claim 1, wherein the suction element comprises a paddle formed on the elongated arm and disposed near a distal end of the suction element.

12. The device of claim 1, comprising:
a paddle having a raised surface on an inner surface thereof.

13. The device of claim 1, comprising:
at least one suction inlet spaced apart from a distal end of a raised surface of the suction element.

14. (canceled)

15. The device of claim 1, comprising:
a clip to releasably couple with the suction port and the irrigation port and located exterior to the oral cavity during use, the clip configured to control a distance between inner edges of the suction element and the irrigation element when the device is placed within the oral cavity.

16. (canceled)

17. The device of claim 1, comprising:
the suction port and the irrigation port each including a positioning slot for releasably receiving a clip where the positioning slot is located at a distance away from a front surface of a bridge and at least one slot of the clip is located to allow the suction port and the irrigation port to be moved together with the clip placed at the positioning slots thereby widening a distance between inner edges of the suction element and the irrigation element.

18. The device of claim 1, comprising:
a suction line fluidly coupled with the suction port.

19. The device of claim 1, comprising:
an irrigation line fluidly coupled with the irrigation port.

20.-25. (canceled)

26. The device of claim 1, comprising:
a locking mechanism to releasably couple with the suction port and the irrigation port and exterior to the oral cavity during use, the locking mechanism including a first arm having at least one clip and a second arm having at least one ridge, the at least one ridge being releasably engaged by the at least one clip and configured to control a distance between the suction element and the irrigation element with the device placed within the oral cavity.

27.-28. (canceled)

29. The device of claim 1, comprising:
a handle mechanism including male and female connectors, the male and female connectors including first and second tabs, respectively, and the first tab and the second tab are releasably engaged when pressed together to control a distance between the suction element and the irrigation element.

30.-31. (canceled)

32. The device of claim 1, comprising:
the suction element including at least one of a rectangular shaped slot, a conical shaped slot, an elliptical shaped slot, a semi-circular shaped slot, an almond-shaped slot, a curved slot or a curved slit.

33.-49. (canceled)

50. The device of claim 1, comprising:
the elongated arm configured to dispose between a cheek and upper and lower teeth of the patient at the location of the oral cavity.

51. The device of claim 1, wherein the location of the oral cavity is a first location of the oral cavity, comprising:
the plate configured to dispose between upper and lower teeth of the patent at a second location of the oral cavity.

52. The device of claim 1, comprising:
the suction element including a paddle for placement between a check and upper and lower teeth of the patient at the location of the oral cavity.

53. The device of claim 1, comprising:
the suction port fluidically coupled with the suction element to provide negative pressure thereto to suction at least the first portion of the oral cavity.
